(19) United States
(12) Patent Application Publication
(10) Pub. No.: US 2024/0309329 A1
(43) Pub. Date: Sep. 19, 2024

(54) MESENCHYMAL STEM CELL-DERIVED EXOSOME DRUG DELIVERY FOR CANCER AND OTHER DISORDERS

(71) Applicant: MAM Holdings of West Florida, L.L.C., Palm Harbor, FL (US)

(72) Inventor: Carl Randall Harrell, Tarpon Springs, FL (US)

(21) Appl. No.: 18/607,107

(22) Filed: Mar. 15, 2024

Related U.S. Application Data
(60) Provisional application No. 63/452,959, filed on Mar. 17, 2023.

(51) Int. Cl. C12N 5/0775; A61K 9/50; A61K 45/06

(52) U.S. Cl. CPC C12N 5/0662; A61K 9/5068; A61K 45/06; C12N 2500/02

(57) ABSTRACT

The present invention relates generally to methods and compositions for immunotherapy and drug delivery. In particular, the present invention relates to methods of producing exosomes from mesenchymal stem cells and, optionally, loading said exosomes with one or more bioactive substances. The exosomes may be loaded using electroporation with one or more bioactive substances such as proteins, miRNAs, and/or siRNA. In specific embodiments, the exosomes may be provided to an individual in need thereof, including where the individual in need thereof is an individual having a medical disorder such as cancer and other disorders.

### CROSS-REFERENCE TO RELATED APPLICATION

This application claims priority to U.S. Provisional Application No. 63/452,959, filed Mar. 17, 2023, which is hereby incorporated by reference in its entirety.

### FIELD OF THE DISCLOSURE

The present invention relates generally to methods and compositions for immunotherapy and drug delivery. In particular, the present invention relates to methods of producing exosomes from mesenchymal stem cells and, optionally, loading said exosomes with one or more bioactive substances.

### BACKGROUND

Mesenchymal stem cells (MSCs) are multi-functional stem cells that are present in multiple human tissues. They can be found in the spinal cord, umbilical cord blood, umbilical cord tissue, placenta tissue, adipose tissue, and the like. With low immunogenicity, multi-directional differentiation ability, in particular homing ability, MSCs have significant potential in various diseases including cancers, cardiovascular diseases, nervous diseases, hematopoietic diseases, and the like.

Exosomes derived from mesenchymal stem cells (MSC-Exos) are nano-sized extracellular vesicles enriched with bioactive molecules (e.g., microRNAs, enzymes, cytokines, chemokines, immunomodulatory, trophic, and growth factors), that regulate survival, phenotype and function of malignant and tumor-infiltrated immune cells. Due to their nano-sized dimensions and bilayer lipid envelope, MSC-Exos easily bypass biological barriers and may serve as drug carriers to deliver bioactive substances such as chemotherapeutics directly into tumor cells. A lipid bilayer maintains the integrity of exosomes and stabilizes biological activities. Protein modification on the surface enhances the recognition and targeting ability of exosomes. MSC-Exos have many unique characteristics, such as small size, low immunogenicity, long-circulating half-life, good penetration, and good biocompatibility. It is one of the best choices for researchers to find drug carriers in vivo. In prior studies, researchers have used MSC-Exos as a carrier to deliver RNA, protein, and molecular drugs to specific parts of the body to achieve targeted therapy.

Mesenchymal stem cells (“MSC”) and MSC-Exos have potent immunosuppressive properties and their therapeutic potential in the alleviation of autoimmune diseases and select cancers have been demonstrated in experimental and clinical trials. Amniotic fluid-derived MSCs (“AF-MSCs”) exhibit an increased proliferation rate and greater immunosuppressive potential than bone marrow derived MSCs (“BM-MSCs”). Both AF-MSCs and placental tissue-derived mesenchymal stem cells (“PL-MSCs”) contain a variety of biological factors including carbohydrates, proteins and peptides, lipids, lactate, pyruvate, electrolytes, enzymes, and hormones. Notably PL-MSCs are sometimes referred to as umbilical cord (UC)-derived mesenchymal stem cells (UC-MSCs) in the art. In addition, AF-MSCs and PL-MSCs contain many important growth factors including epidermal growth factor (“EGF”), transforming growth factor alpha (“TGF-α”), transforming growth factor beta-1, insulin-like growth factor I (“IGF-I”), and erythropoietin (“EPO”).

AF-MSCs and certain MSC-Exos are believed to have antitumor effects and are preferred for their properties, such as immune-modulating capacity and ability to accumulate at the tumor site. Furthermore, AF-MSCs are known to have a scalable capacity for the mass production of exosomes and other immunomodulatory cells for drug delivery. Apart from regulating tumor cell fate, AF-MSC-derived exosomes are capable of being applied for delivery of anticancer therapeutics. For example, peptides conjugated on the surface of MSC-Exos can improve the targeting of MSC-Exos in various cancer-related applications. In principle, targeting peptides may be applied to research breast cancer, lung cancer, liver cancer, heart disease, and brain disease.

AF-MSCs-derived biological products present numerous benefits as bioactive substances compared to cells or synthetic nanoparticles including the potential to be engineered and exceptional biocompatibility/stability features. However, problems such as carrier separation and purification, transportation, drug loading, and targeting still exist and have been well-documented in the literature.

### SUMMARY

In some embodiments, the present inventors disclose method, systems, and compositions for production and use of exosomes, optionally wherein the exosomes are loaded with one or more bioactive substances. In particular embodiments, the disclosure concerns systems, methods, and compositions for production of exosomes to be used as a treatment or as part of a treatment, including as a bioactive substance such as a chemotherapeutic and/or as a delivery to an individual in need thereof.

In some embodiments, the present invention includes a composition for delivering target specific exosomes to the cytoplasm of a tumor cell, wherein the exosomes modulate angiogenesis. In embodiments, the composition comprises an exosome, a bioactive substance, and/or a plasmid. In other embodiments, the exosome is isolated from autologous cells of a subject, from a cell line, from a primary cell culture, and/or from a mesenchymal stem cell. In other embodiments, the at least one plasmid is an RNA plasmid, a DNA plasmid, or any combination thereof.

Also disclosed, in some embodiments, are methods of treating any medical disorder for which the exosomes, optionally loaded with one or more bioactive substances, would be therapeutic. In some embodiments, the exosomes, optionally loaded with one or more bioactive substances such as MSC-Exos, can both treat a disease or a condition in an individual and protect against toxicities associated with other treatments for said disease or condition administered to the individual. In certain embodiments, exosomes are produced from particular cells using multiple agents in the production method of the exosomes. Such exosomes may be produced from particular cells, including at least stem cells, and for example, mesenchymal stem cells (MSCs, which may also be referred to as mesenchymal stromal cells). The MSCs may be derived from any suitable tissue, but in a specific case they are derived from umbilical cord tissue and/or amniotic fluid. Such exosomes may be modified to harbor one or more bioactive substances, and in some cases, the exosomes are electroporated to be made to harbor one or more bioactive substances.

In some embodiments, the umbilical MSCs are from cord tissue, bone marrow, adipose tissue, dental tissue, placental tissue, amniotic fluid, synovial fluid, peripheral blood, Wharton's Jelly, umbilical cord blood, skin tissue, liver tissue, lung tissue, blood vessels, salivary glands, skeletal muscle, mammary gland, or a mixture thereof. In some embodiments, the MSCs are from umbilical cord tissue or amniotic fluid. In some embodiments, the one or more bioactive substances is miRNA, siRNA, shRNA, protein, peptides, drug, lipids, DNA, RNA, or a combination thereof. In some embodiments, the one or more bioactive substances is protein, peptides, drugs, and/or lipids, and wherein the concentration of the protein, peptides, drugs, and/or lipids is between 1 μg/mL and 1000 mg/mL. In some embodiments, the protein comprises an antibody or antibody fragment. In some embodiments, the one or more bioactive substances is miRNA, MSC-Exos, a nucleic acid therapeutic, and/or a protein therapeutic. Notably, synovial fluid is a clear, thick liquid that acts as a lubricant and cushion in joints. It helps to reduce friction between bones and provides nutrients to the cartilage in the joint. It is produced and maintained by the synovium, which is the soft tissue lining the joint capsule.

### BRIEF DESCRIPTION OF THE DRAWINGS

The drawings form part of the present specification and are included to further demonstrate certain embodiments of the present disclosure. The disclosure may be better understood by reference to one or more of these figures in combination with the detailed description of specific embodiments presented herein.

FIG. 1 shows various molecular mechanisms responsible for beneficial effects of MSC-Exos in a muscle cell.

Reference to various embodiments does not limit the scope of the invention. FIGURES represented herein are not limitations to the various embodiments according to the invention and are presented for exemplary illustration of the invention.

### DETAILED DESCRIPTION

The detailed description set forth below in connection with the appended drawings is intended as a description of presently-preferred embodiments of the invention and is not intended to represent the only forms in which the present invention may be constructed or utilized. The description sets forth the functions and the sequence of steps for constructing and operating the invention in connection with the illustrated embodiments. It is to be understood, however, that the same or equivalent functions and sequences may be accomplished by different embodiments that are also intended to be encompassed within the spirit and scope of the invention.

Unless otherwise noted, technical terms are used according to conventional usage. Aspects of the disclosed methods employ, unless indicated specifically to the contrary, conventional methods of chemistry, biochemistry, organic chemistry, molecular biology, microbiology, recombinant DNA techniques, genetics, immunology, and cell biology, many of which are described below for the purpose of illustration. Such techniques are explained fully in the literature. Definitions of common terms in molecular biology may be found in Lewin's Genes X, ed. Krebs et al., Jones and Bartlett Publishers, 2009 (ISBN 0763766321); George P. Rédei, Encyclopedic Dictionary of Genetics, Genomics, Proteomics and Informatics, 3^{rd} Edition, Springer, 2008 (ISBN: 1402067534); Ausubel et al., Current Protocols in Molecular Biology (John Wiley and Sons, updated July 2008); Short Protocols in Molecular Biology: A Compendium of Methods from Current Protocols in Molecular Biology, Greene Pub. Associates and Wiley-Interscience; Glover, DNA Cloning: A Practical Approach, vol. I & II (IRL Press, Oxford, 1985); Anand, Techniques for the Analysis of Complex Genomes, (Academic Press, New York, 1992); Transcription and Translation (B. Hames & S. Higgins, Eds., 1984); Perbal, A Practical Guide to Molecular Cloning (1984); Kendrew et al. (eds.), The Encyclopedia of Molecular Biology, published by Blackwell Publishers, 1994 (ISBN 0632021829); Robert A. Meyers (ed.), Molecular Biology and Biotechnology: a Comprehensive Desk Reference, published by Wiley, John & Sons, Inc., 1995 (ISBN 0471186341); Harlow and Lane, Antibodies, (Cold Spring Harbor Laboratory Press, Cold Spring Harbor, N.Y., 1998) Current Protocols in Immunology Q. E. Coligan, A. M. Kruisbeek, D. H. Margulies, E. M. Shevach and W. Strober, eds., 1991); Annual Review of Immunology; as well as monographs in journals such as Advances in Immunology, and other similar references.

### I. Definitions

As used herein, the singular forms “a,” “an,” and “the,” may refer to both the singular as well as plural, unless the context clearly indicates otherwise. As used herein, the term “comprises” can mean “includes.” Thus, “comprising a cancer-associated immune cell” may mean “including a cancer-associated immune cell” without excluding other elements. Although many methods and materials similar or equivalent to those described herein can be used, particular suitable methods and materials are described below. In case of conflict, the present specification, including explanations of terms, will control. In addition, the materials, methods, and examples are illustrative only and not intended to be limiting. All references, including patent applications and patents are herein incorporated by reference. The use of the word “a” or “an” when used in conjunction with the term “comprising” may mean “one,” but it is also consistent with the meaning of “one or more,” “at least one,” and “one or more than one.”

Throughout this application, the terms “about,” “substantially,” and “approximately” are used to indicate that a value includes the inherent variation of error for the measurement or quantitation method or degree of variability in a value or range. Thus, the terms “about,” “substantially,” and “approximately” mean, in general, the stated value plus or minus 5%. The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed disclosure. As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that aspects described herein in the context of the term “comprising” may also be implemented in the context of the term “consisting of” or “consisting essentially of” Throughout this specification, unless the context requires otherwise, the words “comprise”, “comprises” and “comprising” will be understood to imply the inclusion of a stated step or element or group of steps or elements but not the exclusion of any other step or element or group of steps or elements.

Throughout this application, “consisting of” means including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Any method in the context of a therapeutic, diagnostic, or physiologic purpose or effect may also be described in “use” claim language such as “use of” any compound, composition, or agent discussed herein for achieving or implementing a described therapeutic, diagnostic, or physiologic purpose or effect.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Reference throughout this specification to “one aspect,” “an aspect,” “a particular aspect,” “a related aspect,” “a certain aspect,” “an additional aspect,” or “a further aspect” or combinations thereof means that a particular feature, structure or characteristic described in connection with the aspect is included in at least one aspect of the present disclosure. Thus, the appearances of the foregoing phrases in various places throughout this specification are not necessarily all referring to the same aspect. Furthermore, the particular features, structures, or characteristics may be combined in any suitable manner in one or more aspects.

The term “therapeutically effective amount” refers to an amount sufficient to produce a desired therapeutic result, for example an amount of exosomes sufficient to improve at least one symptom of a medical condition in a subject to whom the cells are administered.

“Individual, “subject,” and “patient” are used interchangeably and can refer to either a human or non-human, such as primates, mammals, and vertebrates. In particular aspects, the subject is a human. The subject is of any age, gender, or race. The subject can be a patient, e.g., have or be suspected of having a disease (that may be referred to as a medical condition), such as benign or malignant cancer, an auto- or allo-immune condition, an infectious disease, a tissue injury, a skin disorder, or a wound. The subject may be undergoing or have undergone treatment. The subject may be asymptomatic. The subject may be a healthy individual desirous of prevention of a disease or condition. The term “subject/Individual/Patient” also refers to a vertebrate, such as a mammal, for example a human. Mammals include, but are not limited to, murines, simians, humans, farm animals, sport animals, and pets. In one embodiment, the subject is a non-human mammalian subject, such as a monkey or other non-human primate, mouse, rat, rabbit, guinea pig, pig, goat, sheep, dog, cat, horse, or cow. In some examples, the subject is a laboratory animal/organism, such as a mouse, rabbit, guinea pig, or rat. In one example a subject includes farm animals and domestic animals or pets (such as a cat or dog). In one example, a subject is a human patient that has a cancer, has been diagnosed with a cancer, or are at risk or having a cancer.

As used herein, “treat,” “treating,” or “treatment” or equivalent terminology refer to both therapeutic treatment and prophylactic or preventative measures, wherein the object is to prevent or slow down (lessen) an undesired physiological change or disorder, such as the growth, development, or spread of a tumor or infectious disease, tumor relapse, a manifestation of an auto- or allo-immune disorder, a manifestation of a tissue injury, a manifestation of a skin disorder, or a wound. For purposes of this disclosure, beneficial or desired clinical results include, but are not limited to, alleviation or amelioration of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. “Treatment” does not necessarily indicate complete eradication or cure of the disease or condition, or associated symptoms thereof. Those in need of treatment include those already with the condition or disorder as well as those prone to have the condition or disorder or those in which the condition or disorder is to be prevented. The results of treatment can be determined by methods known in the art, such as determination of reduction of, e.g., tumor burden or viral load, determination of restoration of function, or other methods known in the art. The term “treating, treatment, and therapy” further refers to any success or indicia of success in the attenuation or amelioration of an injury, pathology or condition, including any objective or subjective parameter such as abatement, remission, diminishing of symptoms or making the condition more tolerable to the patient, slowing in the rate of degeneration or decline, making the final point of degeneration less debilitating, improving a subject's physical or mental well-being, or prolonging the length of survival. Treatment does not necessarily indicate complete eradication or cure of the disease or condition, or associated symptoms thereof. The treatment may be assessed by objective or subjective parameters; including the results of a physical examination, blood and other clinical tests (such as imaging), and the like. In some examples, treatment with the disclosed methods results in a decrease in the number, volume, and/or weight of a tumor and/or metastases.

As used herein, “prevent,” and similar words such as “prevented,” “preventing,” etc., indicate an approach for preventing, inhibiting, or reducing the likelihood of the occurrence or recurrence of, a disease or condition, e.g., cancer, an infectious disease, an auto- or allo-immune disorder, a tissue injury, a skin disorder, or a wound. It also refers to delaying the onset or recurrence of a disease or condition or delaying the occurrence or recurrence of the symptoms of a disease or condition. As used herein, “prevention” and similar words also includes reducing the intensity, effect, symptoms and/or burden of a disease or condition prior to onset or recurrence of the disease or condition.

The term “therapeutic benefit” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may include but is not limited to a reduction in the size of a tumor, a reduction in the invasiveness of a tumor, reduction in the growth rate of the cancer, or prevention of metastasis. Treatment of cancer may also refer to prolonging survival of a subject with cancer. Treatment of an infectious disease may include but is not limited to a reduction in the spread of an infectious disease in an individual, a reduction in the invasiveness of an infectious disease, reduction in the rate of transmission of an infectious disease, or prevention of spread of an infectious disease. Treatment of infectious disease may also refer to prolonging survival of a subject with an infectious disease. Treatment of a cancer, an auto- or allo-immune disorder, or other disorders may include but is not limited to a reduction in pain, edema, elevated temperature, and/or inflammation or prevention of immune rejection. Treatment of cancer, an auto- or allo-immune disorder, or other disorders may also refer to prolonging survival of a subject with cancer, an auto- or allo-immune disorder, or other disorders. Treatment of a tissue injury may include but is not limited to a reduction in the spread of a tissue injury in an individual or a reduction in the pain or inflammation associated with a tissue injury. Treatment of a tissue injury may also refer to prolonging survival of a subject with a tissue injury.

The term “effective amount/therapeutically effective amount”) refers to the amount of an agent (e.g., a chemotherapeutic or MSC-Exos formulation disclosed herein, or other anti-cancer agents) that is sufficient to effect beneficial or desired therapeutic result, including clinical results. An effective amount may vary depending upon one or more of: the subject and disease condition being treated, the sex, weight and age of the subject, the severity of the disease condition, the manner of administration, the ability of the MSC-Exos and MSC-Exos, for example, formulations to elicit a desired response in the individual, and the like. The beneficial therapeutic effect can include enablement of diagnostic determinations; prevention of disease or tumor formation; amelioration of a disease, symptom, disorder, or pathological condition; reducing or preventing the onset of a disease, symptom, disorder or condition; and generally counteracting a disease, symptom, disorder or pathological condition. The term “effective amount” includes an amount that is effective to “treat” a subject (e.g., a patient or subject). When a therapeutic amount is indicated, the precise amount of the compositions of the present disclosure to be administered can be determined by a physician with consideration of individual differences in age, weight, tumor size, extent of infection or metastasis, and condition of the patient (subject).

In one embodiment, an “effective amount” (e.g., of a chemotherapy agent, MSC-Exos, a costimulatory molecule, or ionizing radiation) may be an amount sufficient to increase the rate of survival of a subject, reduce the volume/size of a tumor, the weight of a tumor, the number/extent of metastases, reduce the volume/size of a metastasis, the weight of a metastasis, or combinations thereof, for example by at least about 10%, at least about 20%, at least about 25%, at least about 50%, at least about 70%, at least about 75%, at least about 80%, at least about 90%, at least about 95%, or at least about 99% (as compared to no administration of the bioactive substance). In one embodiment, an “effective amount” (e.g., of MSC-Exos, or ionizing radiation described herein) may be an amount sufficient to increase the survival time of a subject, such as a subject with cancer, for example by at least about 10%, at least about 20%, at least about 25%, at least about 50%, at least about 70%, at least about 75%, at least about 80%, at least about 90%, at least about 95%, 100%, 200%, 300%, 400%, or 500% (as compared to no administration of the bioactive substance).

The phrases “pharmaceutical or pharmacologically acceptable” refers to molecular entities and compositions that do not produce an adverse, allergic, or other untoward reaction when administered to an animal, such as a human, as appropriate. The preparation of a pharmaceutical composition comprising an antibody or additional active ingredient will be known to those of skill in the art in light of the present disclosure.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

The term “active agent,” refers to a physiologically or pharmacologically active substance that acts locally and/or systemically in the body. An active agent is a substance that is administered to an individual for the treatment (e.g., bioactive substance, cancer bioactive substance, and the like), prevention (e.g., prophylactic agent), or diagnosis (e.g., diagnostic agent) of a disease or disorder. Active agents may also include therapeutics that prevent or alleviate symptoms such as symptoms associated with breast cancer or related treatments. Active agents may be a subset of “bioactive substances”.

The term “administering” or “administration” refers to providing or giving a subject an agent or formulation, such as MSC-Exos or another cancer prophylactic or anti-cancer bioactive substance, by any effective route. Exemplary routes of administration include, but are not limited to, injection (such as subcutaneous, subdermal, intramuscular, intradermal, intraperitoneal, intracerebroventricular, intraosseous, intratumoral, intraprostatic, and intravenous), transdermal, intranasal, oral, vaginal, rectal, and inhalation routes.

The term “amniotic factor,” generally refers to molecules naturally present in the amniotic fluid. These include carbohydrates, proteins and peptides such as enzymes and hormones, lipids, metabolic substrates and products such as lactate and pyruvate, and electrolytes.

The term “antigen” refers to a compound, composition, or substance that can stimulate the production of antibodies or an immune response in an animal, including compositions (such as one that includes a tumor-specific protein) that are injected or absorbed into an animal. An antigen reacts with the products of specific humoral or cellular immunity, including those induced by heterologous antigens, such as the disclosed antigens.

The term “peptide” refers to a polymer of amino acid residues. “Polypeptide,” “peptide”, and “protein” are used interchangeably herein. The terms apply to amino acid polymers of one or more amino acid residues, an artificial chemical mimetic of a corresponding naturally occurring amino acid, naturally occurring amino acid polymers, and non-naturally occurring amino acid polymers.

The term “cancer” refers to a class of diseases or conditions in which abnormal cells divide without control and can invade nearby tissues. A malignant cancer is one in which a group of tumor cells display one or more of uncontrolled growth (e.g., division beyond normal limits), invasion (e.g., intrusion on and destruction of adjacent tissues), and metastasis (e.g., spread to other locations in the body via lymph or blood). As used herein, the term “metastasize” refers to the spread of cancer from one part of the body to another. A tumor formed by cells that have spread is called a “metastatic tumor” or a “metastasis.” The metastatic tumor contains cells that are like those in the original (primary) tumor. A “cancer cell” or “tumor cell” refers to an individual cell of a cancerous growth or tissue. A tumor refers generally to a swelling or lesion formed by an abnormal growth of cells, which may be benign, pre-malignant, or malignant. Most cancers form tumors, but some, e.g., leukemia, and some blood cancers, do not necessarily form tumors. For those cancers that form tumors, the terms cancer (cell) and tumor (cell) are used interchangeably. The amount of a tumor in an individual is the “tumor burden” which can be measured as the number, volume, or weight of the tumor.

Exemplary tumors, such as “cancers”, that can be treated using the disclosed MSC-Exos formulations include solid tumors, such as breast carcinomas (e.g. lobular and duct carcinomas, such as a triple negative breast cancer), sarcomas, carcinomas of the lung (e.g., non-small cell carcinoma, large cell carcinoma, blood cancers, squamous carcinoma, and adenocarcinoma), mesothelioma of the lung, colorectal adenocarcinoma, stomach carcinoma, prostatic adenocarcinoma, ovarian carcinoma (such as serous cystadenocarcinoma and mucinous cystadenocarcinoma), ovarian germ cell tumors, testicular carcinomas and germ cell tumors, pancreatic adenocarcinoma, biliary adenocarcinoma, hepatocellular carcinoma, bladder carcinoma (including, for instance, transitional cell carcinoma, adenocarcinoma, and squamous carcinoma), renal cell adenocarcinoma, endometrial carcinomas (including, e.g., adenocarcinomas and mixed Mullerian tumors (carcinosarcomas)), carcinomas of the endocervix, ectocervix, and vagina (such as adenocarcinoma and squamous carcinoma of each of same), tumors of the skin (e.g., squamous cell carcinoma, basal cell carcinoma, malignant melanoma, skin appendage tumors, Kaposi sarcoma, cutaneous lymphoma, skin adnexal tumors and various types of sarcomas and Merkel cell carcinoma), esophageal carcinoma, carcinomas of the nasopharynx and oropharynx (including squamous carcinoma and adenocarcinomas of same), salivary gland carcinomas, brain and central nervous system tumors (including, for example, tumors of glial, neuronal, and meningeal origin), tumors of peripheral nerve, soft tissue sarcomas and sarcomas of bone and cartilage, head and neck squamous cell carcinoma, and lymphatic tumors (including B-cell and T-cell malignant lymphoma). In one example, the tumor is an adenocarcinoma. In another example, the cancer is pancreatic adenocarcinoma. In yet another example, the cancer is colorectal adenocarcinoma. The disclosed MSC-Exos formulations can also be used to treat liquid tumors, such as a lymphatic, white blood cell, or other type of leukemia. In a specific example, the tumor treated is a tumor of the blood, such as a leukemia (for example acute lymphoblastic leukemia (ALL), chronic lymphocytic leukemia (CLL), acute myelogenous leukemia (AML), chronic myelogenous leukemia (CML), hairy cell leukemia (HCL), T-cell prolymphocytic leukemia (T-PLL), large granular lymphocytic leukemia, and adult T-cell leukemia), a lymphoma (such as Hodgkin's lymphoma or non-Hodgkin's lymphoma), or a myeloma.

The term “decrease/lower/lessen/reduce/abate” refers generally to the ability of a composition contemplated herein (e.g., MSC-EXO-delivered chemotherapy, anti-cancer MSC-Exos, or the like) to produce, elicit, or cause a lesser physiological response (i.e., downstream effects) compared to the response caused by either vehicle or a control molecule/composition. A “decrease” or “reduced” amount is typically a “statistically significant” amount, and may include an decrease that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response (reference response) produced by vehicle, a control composition, or the response in a particular cell lineage.

The term “enhance/induce/increase” refers generally to the ability of a composition contemplated herein to produce, elicit, or cause a greater physiological response (i.e., downstream effects) compared to the response caused by either vehicle or a control molecule/composition. A measurable physiological response may include inducing response of cancer-associated endogenous immune cells in the subject and/or an increase in cancer cell death killing ability, among others apparent from the understanding in the art and the description herein. An “enhanced” or “increased” amount is typically a “statistically significant” amount, and may include an increase that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response produced by vehicle or a control composition.

The term “enteral administration” means administration via absorption through the gastrointestinal tract. Enteral administration can include oral and sublingual administration, gastric administration, or rectal administration.

The term “growth factors,” refers to a group of proteins or hormones that stimulate the cellular growth. Growth factors play an important role in promoting cellular differentiation and cell division, and they occur in a wide range of organisms.

The term “immune cell” refers to any cell of the immune system that has one or more effector functions (e.g., cytotoxic cell killing activity, secretion of cytokines, induction of ADCC and/or CDC).

The terms “immunologic”, “immunological” or “immune” response is the development of a beneficial humoral (antibody mediated) and/or a cellular (mediated by antigen-specific T cells or their secretion products) response directed against an immunogen in a recipient patient. Such a response can be an active response induced by administration of immunogen or a passive response induced by administration of antibody or primed T-cells. A cellular immune response is elicited by the presentation of polypeptide epitopes in association with Class I or Class II MHC molecules to activate antigen-specific CD4+T helper cells and/or cos+ cytotoxic T cells. The response may also involve activation of monocytes, macrophages, NK cells, basophils, dendritic cells, astrocytes, microglia cells, eosinophils or other components of innate immunity. The presence of a cell-mediated immunological response can be determined by proliferation assays (CD4+ T cells) or CTL (cytotoxic T lymphocyte) assays. The relative contributions of humoral and cellular responses to the protective or therapeutic effect of an immunogen can be distinguished by separately isolating antibodies and T-cells from an immunized syngeneic animal and measuring protective or therapeutic effect in a second subject.

The term “ionizing radiation” refers to radiation, traveling as a particle or electromagnetic wave, that carries sufficient energy to detach electrons from atoms or molecules, thereby ionizing an atom or a molecule. Ionizing radiation is made up of energetic subatomic particles, ions or atoms moving at high speeds and electromagnetic waves on the high-energy end of the electromagnetic spectrum. Radiation has been demonstrated to induce adaptive immune responses to mediate tumor regression. In addition, the induction of type I IFNs by radiation is essential for the function of CD8+ T cells. Radiation induces cell stress and causes excess DNA breaks, indicating that nucleic acid-sensing pathway likely account for the induction of type I IFNs upon radiation. Type I IFN responses in DCs dictate the efficacy of antitumor radiation. In contrast, chemotherapeutic substances and anti-HER2 antibody treatments have been demonstrated to depend on a distinct immune mechanism to trigger adaptive immune responses. In general, therapeutic radiation-mediated antitumor immunity depends on a proper cytosolic DNA sensing pathway. In embodiments, MSC-Exos is administered in combination with radiation therapy.

Any limitation discussed with respect to one aspect of the disclosure may apply to any other aspect of the disclosure. Furthermore, any composition of the disclosure may be used in any method of the disclosure, and any method of the disclosure may be used to produce or to utilize any composition of the disclosure. Aspects of an embodiment set forth in the Examples are also aspects that may be implemented in the context of aspects discussed elsewhere in a different Example or elsewhere in the application, such as in the Summary, Detailed Description, Claims, and Description of the Drawings. Other objects, features and advantages of the present disclosure will become apparent from the following detailed description. It should be understood, however, that the detailed description and the specific examples, while indicating specific aspects of the disclosure, are given by way of illustration only, since various changes and modifications within the spirit and scope of the disclosure will become apparent to those skilled in the art from this detailed description.

The instant disclosure provides specific systems, methods, and compositions for producing exosomes from mesenchymal stem cells (MSCs) and for loading said exosomes with bioactive substances. As shown herein, MSC-derived exosomes prepared according to the disclosed procedures are stable and bioactive, and electroporation of these MSC-derived exosomes can be used to produce exosomes loaded with potent therapeutics such as chemotherapeutic substances, MSC-Exos, protein therapeutics, nucleic acid therapeutics, and the like.

In embodiments, placental tissue-derived mesenchymal stem cells (“PL-MSCs”), also known as umbilical cord (UC)-derived mesenchymal stem cells (UC-MSCs), produce significantly higher numbers of exosomes compared with bone marrow (BM)-derived MSCs (BM-MSCs). Additionally, like BM-MSC-derived exosomes, PL-MSC-derived exosomes (PL-EXOs) home to tumors in vivo. Using electroporation, these exosomes can be directly loaded with bioactive substances, including but not limited to proteins, nucleic acids and small molecular drugs. This strategy is advantageous because large volume production is more feasible than methods involving transduction of MSCs with lentivirus (LV) to express some bioactive substances, and the reproducibility of the amount of therapeutic in each exosome is highly controlled, rendering this method more scalable and more “drug-like” than the LV-transduction method, in at least some aspects. Additionally, this strategy separates the production/isolation of the exosomes from the loading with bioactive substance, allowing for quality control of each stage.

In embodiments, the disclosed methods are practical, efficient, and allow for the clinical use of exosomes as bioactive substances for, as examples: the treatment of patients with solid and liquid tumors, as well as toxicities associated therewith; the treatment of patients with alloimmune or autoimmune disorders; the treatment of patients with microbial infections; the treatment of patients with skin disorders; the treatment of wounds; vehicles for gene and drug delivery; and bioactive substances for regenerative and/or reparative medicine settings.

### II. Exosome-Based Cancer Therapy

Optimal tumor growth and metastasis require generation of a specific microenvironment that enables enhanced viability, proliferation, invasion and migration of malignant cells. Mesenchymal stem cells (MSC) and MSC-Exos modulate tumor microenvironments and are therefore ideal vehicles for delivery of bioactive substances to various tumors. In embodiments, the present inventors disclose target specific exosomes derived from umbilical cord blood, umbilical cord tissue, bone marrow, adipose tissue, dental tissue, placental tissue, peripheral blood, Wharton's Jelly, skin tissue, or a mixtures thereof.

Advantages of exosomes for cancer therapies include the fact that, unlike synthetic nanoparticles, exosomes are more biocompatible and biodegradable, and thus have low toxicity and immunogenicity. Although other cell-derived EVs are also biocompatible, they are bigger than exosomes and more heterogeneous which limited their application for drug loading and delivery. As described above, exosomes can also be easily generated because most cell types can produce exosomes. Exosomes are stable in biological fluids and their small size enables exosomes to easily escape from lung clearance and pass through the blood-brain barrier. Adherence and internalization of exosomes within tumor cells is 10-times higher than liposomes of a similar size, indicating a higher specificity of exosomes for cancer targeting. In addition, due to enhanced permeability and retention effect, nanometric exosomes tend to accumulate in tumor tissues containing abnormally formed blood vessels than they do in normal tissues, thus exosomes can easily reach the bulk of the solid tumors to increase their drug delivery efficiency. Moreover, exosomes can be engineered with tumor-targeting proteins, peptides, or antibodies for precise drug and therapeutic nucleic acid delivery. The present invention particularly relates to the modulation of tumor angiogenesis and anti-tumor immunity.

### Modulation of Tumor Angiogenesis and Anti-Tumor Immunity

It is known in the art that generation of new blood vessels in rapidly proliferating tumors and restoration of injured capillary network in the irradiated tumors are essential for cancer growth and progression. MSC-Exos are enriched with MSC-sourced angiomodulatory miRNAs and growth factors which regulate expression of genes that controls generation of new blood vessels in the tumor microenvironment. In embodiments, this endogenous enrichment allows for MSC-Exos to be adapted into ideal tumor delivery vehicles.

In embodiments, the present invention relates to a method for delivering exosomes to a tumor by modulating the survival and viability of the target tumor cell. In embodiments, MSC-Exos (e.g., exosomes or target specific exosomes) may deliver bioactive substances such as angiostimulatory proteins (e.g., PD-ECGF, IL-6, Ang1, HGF, and SDF-1, directly into the tumor vasculature to activate various intracellular signaling pathways. In some embodiments, MSC-Exo-sourced PD-ECGF is a thymidine phosphorylase which regulates thymidine homeostasis and enhances DNA synthesis by increasing intranuclear concentration of thymine, leading to increased proliferation of ECs and the generation of new blood vessels in the tumor microenvironment. In another example, MSC-Exos delivered to tumors promote EC proliferation and neovascularization in the tumor microenvironment. In another embodiments, upon binding to its receptors, gp80 and gp130, MSC-sourced IL-6 activates the JAK/STAT and MAPK pathways, resulting in increased EC proliferation.

In other embodiments, the present invention relates to a method for inducing new blood vessel generation in the tumor microenvironment through the use of MSC-Exos. MSC-Exos may deliver angiostimulatory proteins, such as PD-ECGF and IL-6, directly into the tumor vasculature to activate various intracellular signaling pathways in recipient ECs. In some embodiments, MSC-Exo-sourced PD-ECGF regulates thymidine homeostasis and enhances DNA synthesis, leading to increased proliferation of ECs and new blood vessel generation. In other embodiments, MSC-derived IL-6 promotes EC proliferation and neovascularization through the activation of the JAK/STAT and MAPK pathways.

Further disclosed are methods for repairing irradiation-injured blood vessels in a tumor microenvironment using MSC-Exo-sourced Ang1. The method comprises administering MSC-Exo-sourced Ang1 to a subject, whereby the Ang1 induces phosphorylation of Tie2 receptor and activates PI3K in a dose-dependent manner, leading to the activation of the Ang1/Tie2/PI3K signaling pathway and modulation of mTOR activity through AKT-dependent mechanisms, thereby preventing caspase-driven apoptosis of ECs. In embodiments, the MSC-Exos may also contain HGF and SDF-1, which improve the migratory properties of ECs and endothelia progenitor cells (EPCs), allowing for enhanced repair of the irradiation-injured blood vessels. In embodiments, the pro-angiogenic activity of MSC-Exo-derived HGF is elicited through tyrosine phosphorylation of its specific receptor, c-Met, expressed on ECs of newly formed blood vessels, resulting in phosphorylation of several kinases (ERK, JNK, PI3K, and p38MAPK) and enhanced proliferation and migration of ECs, ultimately leading to the restoration of the injured blood vessels. In embodiments, MSC-Exo-sourced SDF-1 also enhances neo-angiogenesis in irradiated tumor tissues by binding to CXCR4 on EPCs and enhancing the migration and recruitment of EPCs to the sites of injury, thereby contributing to the restoration of blood vessel integrity.

Further disclosed is a method for promoting survival and repair of blood vessels in the tumor microenvironment by administering to a subject a composition comprising bioactive substances, including MSC-Exo-sourced Ang1, HGF, and/or SDF-1. Ang1 is a potent anti-apoptotic factor that promotes the survival of ECs. Furthermore, Ang1 activates the Tie2 receptor and PI3K in a dose-dependent manner. This Ang1/Ti2/PI3K signaling pathway elicits AKT-dependent modulation of mTOR activity and prevents caspase-driven apoptosis of ECs.

In embodiments, disclosed are exosomes for tumor delivery further that utilize the fact that MSC-Exo-derived HGF improves the migratory properties of ECs and EPCs, enabling enhanced repair of irradiation-injured blood vessels. HGF exerts its pro-angiogenic activity through tyrosine phosphorylation of its specific receptor, c-Met, which is expressed on ECs of newly formed blood vessels. Once activated, several kinases (ERK, c-Jun N-terminal kinases (JNK), PI3K, and p38MAPK) become phosphorylated in ECs, resulting in enhanced proliferation and migration and restoration of injured blood vessels.

In embodiments, MSC-Exo-sourced SDF-1 enhances neo-angiogenesis in irradiated tumor tissues. In one embodiment, target specific exosomes include MSC-Exos contain 26 pro-angiogenic miRNAs which regulate MSC-EC cross-talk in the tumor microenvironment. In some embodiments, MSC-derived miR-424, miR-30b, and miR-30c are administered to a patient, resulting in tube-like structure formation and sprouting of newly generated blood vessels in the tumor vasculature. In one example, MSC-Exo-sourced miR-424 is administered, inducing increased expression of VEGF in tumor cells. In other embodiments, this increased expression of VEGF in tumor cells leads to the binding of VEGF to VEGFR2, activating phosphoinositide phospholipase C (PLCT) and phosphoinositide 3-kinase (PI3K) in tumor ECs. In some embodiments, PLC7 and PI3K modulate mTOR activity and activate protein kinase C (PKC) and extracellular signal-regulated kinase (ERK), suppressing caspase-dependent apoptosis and promoting cyclinD1 activity in a nuclear factor-κB (NF-κB)-dependent manner, thereby enhancing EC survival in the tumor microenvironment. In other examples, MSC-Exo-sourced miR30b and miR30c regulate VEGF synthesis in tumor cells by controlling Delta-like 4 (DLL4) gene expression. In one embodiment, DLL4 is over-expressed in human cancers and regulates the expression of VEGFR, indirectly controlling VEGF-dependent sprouting of newly generated blood vessels.

In embodiments, further to the above, the present invention includes a composition for delivering target specific exosomes to the cytoplasm of a tumor cell, wherein the exosomes modulate angiogenesis. Said composition may include an exosome and bioactive substances, located within the exosome, comprising at least one plasmid (e.g., plasmid is a RNA plasmid, a DNA plasmid, or any combination thereof). In other embodiments, the exosome is isolated from autologous cells of a patient. In other embodiments, the exosome is isolated from a cell line, a primary cell culture, or a combination thereof. In still other embodiments, the exosome is isolated from a mesenchymal stem cell. In embodiments, the at least one plasmid may be an RNA plasmid, a DNA plasmid, or any combination thereof. In other embodiments, the least one plasmid is a retrovirus or an adeno-associated virus (AAV). Further to the above, in still other embodiments, the at least one plasmid promotes expression of the VEGF gene.

Further to the above, MSC-Exos may also mitigate tumor growth and progression by suppressing VEGF-driven generation of the capillary network in the tumor microenvironment. In some embodiments, MSC-Exo-derived miR-16 suppresses VEGF gene expression in murine mammary carcinoma 4T1 cells and attenuates VEGF-dependent neovascularization in breast cancer. In some embodiments, MSC-Exos does not directly affect the viability and proliferation of 4T1 cells but inhibits their capacity for VEGF production. In one example, the concentration of miR-16 in MSC-Exo-treated 4T1 cells is inversely correlated with the levels of VEGF expression. In embodiments, MSC-Exo-dependent suppression of VEGF synthesis in 4T1 cells results in significantly lower proliferation and migration of ECs which were co-cultured with tumor cells. In embodiments, MSC-Exos-based treatment significantly reduces the size and weight of breast cancers in MSC-Exo-treated subjects. In other examples, MSC-Exos down-regulate expression of VEGF and Ang1 and significantly reduced vascular density in OSS carcinomas, leading to the inhibition of proliferation of OSS cells and reduced growth and progression of OSS carcinomas in a subject.

### Anti-Tumor Immunity

In embodiments, MSC-Exos contain all bioactive substances described herein, including all immunomodulatory factors herein. Thus, MSC-Exos may have various immunoregulatory properties. In some embodiments, MSC-Exos deliver immunosuppressive and immunostimulatory factors in tumor infiltrated T lymphocytes, dendritic cells (DCs), neutrophils, tumor-associated macrophages (TAMs), natural killer (NK) cells and by modifying their phenotype and function, regulate anti-tumor immunity and tumor progression.

In other embodiments, MSC-Exo-sourced IL-10 inhibit activation and anti-tumor properties of DCs, T lymphocytes and TAMs. In one example, MSC-Exo-derived IL-10 induces the generation of a tolerogenic, immunosuppressive phenotype in tumor-infiltrated DCs. These tolerogenic DCs have an immature phenotype and do not optimally produce pro-Th1 and pro-Th17 inflammatory cytokines (TNF-α, IL-12, IL-1β, IL-23), and therefore, are not capable of inducing activation of naïve CD4+ and CD8+ T cells or generating Th1 and Th17 cell-driven anti-tumor immune response.

In other examples, MSC-Exos, in an IL-10-dependent manner, induce alternative activation of TAMs. In alternatively activated TAMs, MSC-Exo-derived IL-10 inhibits the synthesis of inflammatory cytokines (IL-1β, tumor necrosis factor alpha (TNF-α)) and prevents IL-1β and TNF-α-driven recruitment of circulating leukocytes in tumor tissue. In other embodiments, IL-1β and TNF-α bind to their receptors (IL-1R, TNFRs) on the tumor vasculature's ECs, activating a MyD88/MAPK-dependent intracellular cascade and several transcriptional factors (NF-κB, activator protein 1 (AP-1)), which increase the expression of genes responsible for the production of E and P selectins and integrin ligands.

In some embodiments, MSC-Exos contain high levels of MSC-derived IL-1Ra and sTNFRs, which bind to IL-1R and TNF-α and prevent IL-1β and TNF-α-dependent recruitment of leukocytes in tumor tissue. As a result, the concentration of IL-1β and TNF-α is significantly lower in the serum samples of MSC-Exo-treated tumor-bearing subjects, accompanied by reduced presence of activated immune cells in the tumor tissues. In other embodiments, MSC-Exo-derived miR-21-5p induces alternative activation of TAMs, suppresses IL-1β and TNF-α-driven inflammatory response, and inhibits anti-tumor immunity. For example, MSC-Exo-derived miR-21-5p may downregulate the expression of Phosphatase and tensin homolog (PTEN) gene, suppressing the synthesis of Arginase I in TAMs and inhibiting their alternative activation.

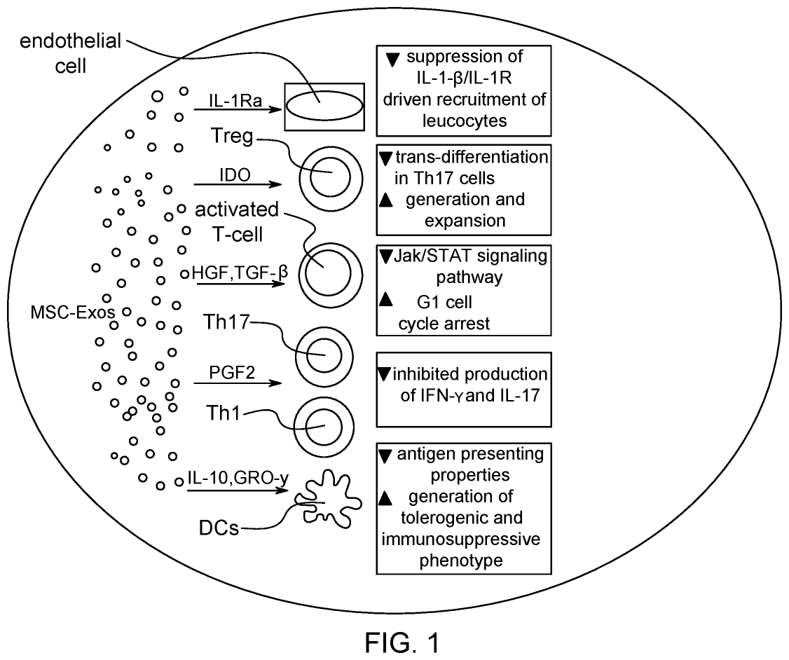

FIG. 1 depicts various effects of MSC-Exos in a muscle cell. In general, the present disclosure relates to drug delivery to various tissue throughout the body, with the exception of delivery to the eye and some internal organs which require special consideration. As shown in FIG. 1, in some embodiments, MSC-Exos sourced IL-1Ra binds to epithelial cells and suppresses IL-1β/IL-1R driven recruitment of leucocytes. Further, FIG. 1 shows that MSC-Exos sourced indoleamine 2,3 dioxygenase (IDO) induces generation and expansion of Tregs and prevents their trans-differentiation in inflammatory Th17 cells. Further, FIG. 1 shows that MSC-derived TGF-β and HGF suppress Jak-Stat signaling pathway and induce G1 cell cycle arrest of activated T cells. In other embodiments, as shown in FIG. 1, MSC-sourced PGE2 reduces production of inflammatory cytokines (IFN-7 and IL-17) in CD4+Th1 and Th17 cells. In other embodiments, as shown in FIG. 1, by delivering IL-10 and GRO-γ, MSC-Exos inhibit DC-dependent antigen presenting properties while inducing generation of tolerogenic and immunosuppressive phenotypes.

In embodiments, MSC-Exos are comprised of enriched TGF-β and PGE2 sourced from MSCs. These components help to prevent the expansion of activated T cells in the tumor microenvironment. TGF-β functions by inhibiting the activation of the JAK-STAT signaling pathway and inducing a G1 cell cycle arrest, which reduces the expansion of activated CD8+T lymphocytes and natural killer cells. In some embodiments, the suppression of cytotoxicity in CTLs and NK cells by MSC-Exos is TGF-β dependent. This suppression enables enhanced tumor growth and progression by reducing the elimination of malignant cells by CTLs and NK cells. Similarly, PGE2 sourced from MSCs also suppresses activated CTL proliferation by down-regulating IL-2 synthesis. Additionally, PGE2 contributes to the reduced antigen presenting properties of tumor-infiltrated dendritic cells and tumor-associated macrophages by decreasing the expression of co-stimulatory molecules and MHC class II proteins.

In other embodiments, the expression of IDO in tumor-infiltrated MSCs is enhanced by inflammatory cytokines such as IFN-7 and TNF-α, which are produced by T lymphocytes and inflammatory TAMs in the tumor microenvironment. IDO converts tryptophan to the immunosuppressive molecule kynurenine, which expands T regulatory cells and reduces the generation of inflammatory CD4+ and CD8+Th17 lymphocytes. In one example, MSC-Exos deliver bioactive substances miR-182 to CD8+ CTLs and NKT cells in the tumors and increased their proliferation and tumor toxicity. In other embodiments, MSC-Exos administered to a cancer patient promote the recruitment of circulating CD4+ and CD8+ T cells and enhance T cell-driven anti-tumor immune response in a CCL2-dependent manner. Thus, in embodiments MSC-Exos have significant immunostimulatory effects on tumor-infiltrated immune cells.

### Dormancy and Resistance

In some embodiments, MSC-Exos modulate the dormancy of malignant cells, which contributes to chemoresistance, metastasis, and recurrence of breast cancer. MSC-Exos regulate the cross-talk between MSCs and dormant breast cancer cells and modulate their resistance to chemotherapeutic drugs. In embodiments, MSC-Exos with the bioactive substance miR-23b induce dormancy of BM2 breast cancer cells and promotes their resistance to docetaxel. In embodiments, MSC-Exos-derived miR-23b further suppresses the expression of the MARCKS gene, which encodes a protein that facilitates cell cycling and motility of BM2 cells.

In other embodiments, MSC-Exo-sourced miR-21-5p induces increased expression of the chemoresistant S100A6 gene in MDA-MB-231 breast cancer cells and promoted their resistance to doxorubicin (DOX) in vitro and in vivo. In line with these findings, MSC-Exo-derived miR-222/223 was found to increase resistance to carboplatin by inducing G0 cell cycle arrest and dormancy of MDA-MB-231 and T47D breast cancer cells. Bliss and co-workers designed a new therapeutic strategy to target dormant breast cancer cells based on these findings. In this study, intravenously infused MSC loaded with antagomiR-222/223 was found to sensitize dormant breast cancer cells to carboplatin-based therapy and significantly increase survival of breast cancer-bearing animals, indicating the potential therapeutic potential of MSC-Exos in the elimination of dormant tumor cells.

In some embodiments, MSC-Exos increase the survival and stemness of cancer stem cells (CSCs) and enhance their resistance to anti-cancer drugs. In one embodiment, MSC-Exos enhance the expression of Oct3/4, NANOG, and SOX2 genes, which regulate the viability and pluripotency of CSCs and promote the expression of the chemoresistant S100A4 gene, which increases their resistance to chemotherapeutic drugs. In one example, target specific MSC-Exos promote the proliferation and decrease the sensitivity of CML cells to tyrosine kinase inhibitors (TKIs). In another embodiments, MSC-Exos-sourced bioactive substance miR-15a induces G0 cell cycle arrest.

The resistance of tumor cells to anti-cancer drugs poses a challenge for effective therapy and impacts patient survival. Researchers have thus focused on determining the molecular mechanisms behind cancer therapy resistance. Many studies have demonstrated that MSC-Exos enhance the resistance of malignant cells to chemotherapy. Disclosed here, MSC-Exos administered to a subject enhance the resistance of gastric cancer cells to 5-fluorouracil (5-FU) by preventing 5-FU-induced apoptosis of these cells. The MSC-Exos activate calcium/calmodulin-dependent protein kinases (CaM-Ks) and Raf/MEK/ERK signaling pathway, which results in increased expression of multi-drug resistance (MDR)-associated genes such as MDR, MRP, and LRP and decreased sensitivity of tumor cells to chemotherapy. In embodiments, MSC-Exos containing miR-23b induce dormancy of BM2 breast cancer cells and promotes their resistance to docetaxel. In embodiments, miR-23b suppresses the expression of the MARCKS gene, encoding a protein that facilitates cell cycling and motility of BM2 cells.

In another embodiment, MSC-Exo-sourced bioactive substance miR-21-5p increases the expression of chemoresistant S100A6 gene in MDA-MB-231 breast cancer cells and promotes their resistance to doxorubicin (DOX) in vitro and in vivo. In further embodiments, MSC-Exo-derived bioactive substance administered to a patient miR-222/223 induces G0 cell cycle arrest and dormancy of MDA-MB-231 and T47D breast cancer cells, increasing their resistance to carboplatin. MSC-Exos also increase the survival and stemness of cancer stem cells (CSCs) and enhance their resistance to anti-cancer drugs. For instance, they enhance the expression of Oct3/4, NANOG, and SOX2 genes, which regulate viability and pluripotency of CSCs and promote the expression of chemoresistant S100A4 gene, increasing the CSCs' resistance to chemotherapeutic drugs.

In embodiments, a xenograft tumor model of chronic myeloid leukemia (CML) is used to show that MSC-Exos promote proliferation and decrease sensitivity of CML cells to tyrosine kinase inhibitors (TKIs). In embodiments, MSC-Exos-sourced miR-15a induces G0 cell cycle arrest, increases synthesis of anti-apoptotic Bcl-2 protein, and suppresses caspase 3-driven apoptosis of CML cells, which attenuates the efficacy of TKIs and increases leukemia progression.

Other siRNA and miRNAs

In some embodiments, MSC-Exos carry bioactive substances such as small interfering RNA (siRNA) for the delivery of siRNA to tumor cells. In one example, GRP78-siRNA-containing MSC-Exos is delivered in hepatocellular carcinoma (HCC) cells to suppress the synthesis of a drug sensitivity-conferring protein and to sensitize the cells to the chemotherapy drug sorafenib. This results in a significant reduction in the viability, proliferative, and invasive properties of the HCC cells. In other embodiments, MSC-Exos carry miRNAs for the induction of miRNA-dependent abrogation of chemoresistance in tumor cells. For instance, miR-122 and miR-199a-overexpressing MSCs may deliver miR-122 and miR-199a-containing exosomes to HCC cells, thereby upregulating apoptosis-related genes and reducing the volume and weight of hepatocellular carcinoma in experimental mice. In other embodiments, MSC-ExosmiR-199a is used to sensitize HCC cells to doxorubicin by inhibiting the mTOR pathway. This results in a suppression of mTOR activity and an increased sensitivity to doxorubicin, leading to a reduction in the growth and progression of hepatocellular carcinoma in doxorubicin-treated tumor-bearing mice.

In embodiments, MSC-Exos carrying bioactive substances are adapted to suppress glioma progression. In some embodiments, this is achieved by overexpressing miR-199a in MSC-Exos, which down-regulates the expression of AGAP2 gene in glioma cells and increases sensitivity to temozolomide (TMZ). In other embodiments, anti-miR-9 and miR-12d-overexpressing MSC-Exos increase chemosensitivity of glioblastoma multiforme (GBM) cells. In one example, MSC-Exo-derived anti-miR-9 suppressed P-glycoprotein expression in GBM cells weakens TMZ resistance and enhances TMZ-driven apoptosis. In other examples, miR-124-overexpressing MSC-Exos suppresses proliferative, migratory, and invasive properties of GBM cells by attenuating CDK6 gene expression.

In further embodiments, MSC-Exos are adapted to suppress non-small cell lung cancer (NSCLC). In some embodiments, this is achieved through overexpression of miR-193a in MSC-Exos, which down-regulates LRRC1 gene expression in NSCLC cells and attenuates cisplatin (DDP) resistance. In other embodiments, results obtained in a murine model of lung cancer show that combined DDP+MSC-Exos therapy is more efficient in suppressing lung cancer growth and progression than DDP-single based treatment. As described above, the MSC-Exos drug delivery mechanism described herein are not relevant to mammalian eyes, which require special considerations.

### III. Systems and Methods of Producing Exosomes

Disclosed herein are systems and methods of producing target specific exosomes (e.g., MSC-Exos, and the like). In specific cases, the present disclosure concerns a manufacturing practice to produce as exosomes. In some embodiments, the exosomes are MSC-derived and produced under particular conditions in combination with being produced from particular cells. In some embodiments, MSCs are from umbilical cord tissue or amniotic fluid, but they can come from any source including, but not limited to, bone marrow, adipose tissue, dental, and placental tissue.

Any step in the process may have a particular media, duration of time, presence of one or more particular gases at specific concentrations, presence or absence of movement (such as rotation), and a combination thereof, for example. In particular aspects, the cells are incubated (e.g., in a cell culture reactor or flask) with media for a particular amount of time, in some cases. This is followed by washing and collection of the cells and exosomes secreted from the cells. The collection of the exosomes (that may be referred to herein as harvesting) may include one step or multiple steps; in cases when the collection of the exosomes occurs more than once, there may or may not be an interval of time by which the exosomes are collected, such as at least, at most, or about 12, 18, 24, 36, 48, 60, 72 hours, or more, or any range or value derivable therein, between collections. The media in which the cells and exosomes are collected may be of a particular kind, and in specific steps when the cells and exosomes are collected the media lacks platelet lysate (PLT-free). In specific cases, the cells are cultured over the course of about 22 hours, and then cells are washed and exosomes secreted from the cells are collected approximately every 48 hours in the EC media-PLT-free (the EC media-PLT free may or may not comprise alpha MEM media supplemented with 2 mM of GLUTAMAX™ (synthetic reagent similar to L-glutamine and that comprises L-alanyl-L-glutamine dipeptide)). These sequential steps may be repeated, such as repeated for a total of 2, 3, 4, or more times.

In specific aspects, the suspension of cells and exosomes are harvested from the system under conditions in which the exosomes produced from the cells consistently have the same or substantially the same markers and physiology. Thus, in specific cases at different times of harvesting, the exosomes are the same or substantially the same by their majority of exosomes having one or more of the same expression markers.

In particular aspects, the process to produce the exosomes occurs in a cell culture reactor, although in alternative cases it does not. In particular aspects, the process to produce the exosomes occurs in flasks. In specific aspects, part or all of the process occurs in a cell culture reactor having controllable conditions that in specific cases may be automated. Although the cell culture reactor may be of any kind, in specific aspects the cell culture reactor comprises a hollow fiber system that may or may not comprise one or more pathways. The multiple porous microchannels comprise inner surfaces suitable for adherence of cells or suitable for modification such that cells may adhere to them, in particular aspects. Alternative cell culture reactor systems include the WAVE CELL CULTURE REACTOR™ (GE Healthcare) or the G-REX® system (Wilson Wolf), as examples.

In certain aspects, the hollow fiber cell culture reactor may be a functionally closed (or semi-closed) system designed for a large-scale cell culture of adherent or non-adherent cells. The system allows the cells to grow (expand in number) in a dynamic environment allowing the continuous perfusion of medium that under suitable conditions mimics particular in vivo intravascular and extravascular compartments in at least some cell culture reactors. That is, in specific cases an intravascular compartment is configured to mimic the intravascular region of the blood system and/or an extravascular compartment is configured to mimic the extravascular hematopoietic system. The hollow fiber system in specific cases comprises hundreds or thousands of semi-permeable pores for the culture of desired cells, including adherent cells. Membranes may make up the inner walls of the porous microchannels and allow exchange of gas and/or nutrients with a homogenous approach, maximizing the growth rate of the cells in a short time. In particular aspects, the process is specifically designed to be suitable for growth of MSCs and to allow for the collection of the exosomes secreted by the cells in a customized method.

Components of the cell culture reactor system comprise vessels and/or compartments for introducing media and/or cells to the system, vessels and/or compartments for expanding the cells (and thereby produce exosomes from the expanding/expanded cells), and vessels and/or compartments for harvesting the cells, the conditioned media comprising the exosomes, and so forth. Examples of compartments for any part of the system include a cell inlet bag, media bag, harvest bag, and waste bag, in specific aspects. The cell culture reactor system utilizes thousands of semi-permeable porous microchannels onto which the cells are adherent, either naturally or because the porous microchannels in the system have been manipulated to allow for adherence of the desired cells. In specific aspects, the system also comprises a gas regulator (that may be referred to as a gas transfer module) that stabilizes desired gas concentrations in the media. Such a gas regulator allows for, if desired, continual infusion of one or more gases into the cell culture reactor.

In specific aspects, the process to produce the desired exosomes utilizes well-defined concentrations of CO₂ (for example, about 5%), O₂ (for example, about 20%), and nitrogen (for example, the conditions are nitrogen balanced).

Appropriate steps are taken to prepare the system prior to loading of the cells, such as preparation of the physical components of the system to facilitate expansion of the cells. The system may be closed or may be semi-closed (as used herein, refers to during the production of exosomes some steps require the opening of the system and the exposure of the sample to the air). Prior to subjecting the cells to be expanded to the system, the cell culture reactor may be subjected to one or more components and/or one or more conditions to facilitate adherence of cells to the cell culture reactor. Cell media may be loaded into the system prior to loading of the cells. For adherent cell production, cells attach and proliferate on the inner surface of each fiber. Suspended cells can be flushed, leaving the adherent cell production for expansion. Automated cell feeding and waste removal means may be part of the system, in specific aspects. In at least some cases, sampling of cells/conditioned media from the system may be provided for without or with interruption of the process. In particular aspects, after cell expansion the adherent cells are released from the hollow fiber walls into suspension, and the suspension including cells and exosomes secreted therefrom are collected.

The exosomes may be separated by any suitable means from the supernatant and cells once the cells are harvested from the process, including from the system. In some cases, there are multiple harvests from the process, and the supernatant, cells, and exosomes from the process may be pooled prior to any further separation or modification steps. In certain cases, exosomes from multiple harvests are processed separately and combined later. In some embodiments, the exosomes are enriched or concentrated following the production process. As one example, the exosomes are separated from cells, cell fragments, and/or larger or smaller vesicles through physical and/or chemical means. In specific cases, the exosomes are concentrated through one or more centrifugations, one or more filtrations (such as ultrafiltration and/or diafiltration), one or more of immunoisolation, chemical precipitation, size exclusion chromatography, microfluidics, or a combination thereof. Different centrifugation steps may occur at different speeds, and/or different filtration steps may occur at different sizes.

In some embodiments, exosomes are enriched or concentrated from the medium of cultured MSCs using differential ultracentrifugation. In some embodiments, differential ultracentrifugation comprises the following steps: 1) the supernatant is centrifuged at 2000×g for 20 minutes, and the pellet comprising cells is discarded; 2) the supernatant is filtrated using at 0.2 pm filter; 3) the supernatant is centrifuged at 100000×g for 240 minutes, and the pellet comprising exosomes and cell proteins is obtained and washed in PBS; and 4) the PBS-washed pellet comprising exosomes and cell proteins is centrifuged at 100000×g for 70-180 minutes, and the pellet comprising exosomes is obtained.

Although differential ultracentrifugation provides reasonably pure exosomes, in some embodiments, an extra purification step is performed using a sucrose cushion. Thus, in some embodiments, exosomes are enriched or concentrated from the medium of cultured MSCs using differential ultracentrifugation followed by filtration through a sucrose gradient. Using a sucrose cushion eliminates more contaminants, such as proteins nonspecifically associated with exosomes, or large protein aggregates, which are sedimented by centrifugation but do not float on a sucrose gradient. Therefore, in some embodiments, the recited differential ultracentrifugation steps further comprise the following steps: 5) resuspend partially purified exosome pellet in PBS total; 6) load Tris/sucrose/heavy water (D20) solution at the bottom of a centrifuge tube, to make a cushion; 7) add the diluted exosomes gently above the sucrose cushion without disturbing the interface, and centrifuge 75 minutes at 100,000×g at 4° C.; 8) with a 5-ml syringe fitted with an 18-G needle, collect ˜3.5 ml of the Tris/sucrose/D20 cushion, which now contains exosomes, from the side of the tube; 9) transfer the exosomes to a fresh ultracentrifuge tube, dilute with PBS, and centrifuge 70 min at 100,000×g, at 4° C.; and 10) resuspend the pellet in PBS.

Exosomes may be used immediately or substantially immediately, or they may be stored prior to use, for example at −80° C. or in liquid nitrogen. In some embodiments, the exosomes are concentrated prior to modification of any kind, whereas in other cases the exosomes are modified prior to concentration. The exosomes may be analyzed following the production process, following the concentration step, and/or during the process itself. Such analysis includes identifying one or more markers, identifying size, determining concentration, determining one or more specific activities for the exosomes (such as migration or immunosuppression, and/or anti-T cell activity) or a combination thereof.

In some embodiments, high concentrations of exosomes are required for effective treatment of one or more diseases or disorders (e.g., cancers, tumor, and the like). In these cases, stem cells are induced to produce an increased amount of exosomes. Exemplary methods for induction of exosomes (“Exos”) from stem cells include treatment of the stem cells with cytokines, treatment with liposome stimulation using one or more stimulant liposomes such as neutral or cationic liposomes (Emam S E et al., Biol Pharm Bull. 2018; 41(5):733-742), or other physical and/or biological methods previously described (Phan J et al., J Extracell Vesicles. 2018; 7(1): 1522236). Generally, methods of isolating exosomes are known including one or more of differential ultracentrifugation-based techniques, size-based techniques, immunoaffinity capture-based techniques, exosome precipitation, and microfluidics-based techniques (Li P et al., Theranostics. 2017; 7(3): 789-804). In some embodiments, the MSC-Exos formulations comprise exogenous exosomes generated ex vivo from amniotic fluid dMSCs, or derived from dMSCs of other sources.

### IV. Exosome Modification

In some embodiments, the exosomes comprise one or more certain characteristics or activities as a result of being produced from MSCs (including particular MSCs, such as from umbilical cord tissue). In other embodiments, the exosomes may be further modified. In particular cases, the exosomes are further modified to harbor (carry) one or more bioactive substances. In some cases, the MSCs are modified (e.g., transfected, transduced, electroporated, etc.), and modified exosomes are generated by the modified MSCs. In some cases, the exosomes themselves are modified (e.g., transfected, transduced, electroporated, etc.).

The modification of the exosomes may occur by any suitable method in the art, but in specific cases the exosomes are loaded with one or more bioactive substances by a vector, electroporation, transfection, using a cationic liposome transfection agent, or a combination thereof. Additionally, or alternatively, in some embodiments, MSCs are modified by any suitable method in the art, but in specific cases the MSCs are loaded with one or more bioactive substances by a vector, electroporation, transfection, using a cationic liposome transfection agent, or a combination thereof, and exosomes comprising the one or more bioactive substances are generated from the modified MSCs. Notably, bioactive substances may also be referred to as agents or therapeutics agents throughout this specification.

The bioactive substance(s) loaded into the exosomes in particular aspects are exogenous with respect to the MSCs. They can be introduced into the exosomes by a number of different techniques. In particular aspects of the disclosure, the exosomes are loaded by electroporation or the use of a transfection reagent.

In specific embodiments, the exosomes are of a specific size such that their size determines the type of bioactive substances that they can carry. In particular cases, the exosomes are 20-500 nm in size, including 30-350, 30-300, 30-250, 30-200, 30-150, 30-100, 30-50, 50-400, 50-350, 50-300, 50-250, 50-200, 50-150, 50-100, 100-400, 100-350, 100-300, 100-250, 100-200, 200-400, 200-350, 200-300, 200-250, 250-400, 250-350, 250-300, 300-400, 300-350, or 350-400 nm in size, or any range or value derivable therein.

### A. Exosome Loading Via Vector, Electroporation, Transfection

In some examples, exosomes are modified by loading the MSCs or exosomes with one or more bioactive substances by a vector, electroporation, transfection using a cationic liposome transfection agent, for example, or a combination thereof.

### 1. Vectors

In one embodiment, exosomes may be loaded by transforming or transfecting the MSCs with a nucleic acid construct that expresses the bioactive substance(s), such that the bioactive substance(s) are present in the exosomes as the exosomes are produced from the cell. In another embodiment, exosomes may also be loaded by directly transforming or transfecting the exosomes with a nucleic acid construct that expresses the bioactive substance(s).

In some embodiments, the nucleic acid construct encoding the bioactive substance(s) is comprised in a vector. In some cases, the nucleic acid construct encoding the bioactive substance(s) is linked to a promoter and incorporated into an expression vector, which is taken up and expressed by cells. The vectors can be suitable for replication and, in some cases, integration in eukaryotes. Typical cloning vectors contain transcription and translation terminators, initiation sequences, and promoters useful for regulation of the expression of the desired nucleic acid sequence. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors. In general, a suitable vector contains an origin of replication functional in at least one organism, a promoter sequence, convenient restriction endonuclease sites, and one or more selectable markers In some embodiments, a suitable vector is capable of crossing the blood-brain barrier.

In certain embodiments the expression vector may be provided to a cell in the form of a viral vector. Viral vector technology is well known in the art and is described, for example, in Sambrook et al. (2001) Molecular Cloning: A Laboratory Manual, Cold Spring Harbor Laboratory, New York), and in other virology and molecular biology manuals. A number of viral based systems have been developed for gene transfer into mammalian cells. Viruses that are useful as vectors include, but are not limited to, retroviruses, adenoviruses, adeno-associated viruses, herpes viruses, and lentiviruses (including self-inactivating lentivirus vectors). For example, adenoviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. Thus, in some embodiments, the nucleic acid encoding the polypeptide sequences is introduced into cells using a recombinant vector such as a viral vector including, for example, a lentivirus, a retrovirus, gamma-retroviruses, an adeno-associated virus (AAV), a herpesvirus, or an adenovirus.

Vectors can also comprise other components or functionalities that further modulate gene delivery and/or gene expression, or that otherwise provide beneficial properties to the targeted cells. Such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide.

Such components also might include markers, such as detectable and/or selection markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. A large variety of such vectors are known in the art and are generally available. When a vector is maintained in a host cell, the vector can either be stably replicated by the cells during mitosis as an autonomous structure, incorporated within the genome of the host cell, or maintained in the host cell's nucleus or cytoplasm.

Eukaryotic expression cassettes included in the vectors particularly contain (in a 5′-to-3′ direction) regulatory elements including a eukaryotic transcriptional promoter operably linked to a protein-coding sequence, splice signals including intervening sequences, a transcriptional termination/polyadenylation sequence, post-transcriptional regulatory elements, and origins of replication.

### 2. Electroporation

In particular embodiments of the disclosure, the MSCs and/or exosomes are loaded by electroporation. As used herein, “electroporation” refers to application of an electrical current or electrical field to facilitate entry of an agent of interest into cells, exosomes, or derivatives thereof. One of skill in the art will understand that any method and technique of electroporation is contemplated by the present disclosure. In some embodiments, an electroporation system may be controlled to create electric current and send it through a cell- or exosome-containing solution. In some embodiments, a static electroporation apparatus is used. In some embodiments, a flow electroporation apparatus is used. In specific embodiments, static or flow electroporation is used with parameters described herein.

The process of electroporation generally involves the formation of pores in a cell membrane, or in an exosome, by the application of electric field pulses across a liquid cell suspension containing cells or exosomes. The pulse induces a transmembrane potential that causes the reversible breakdown of the cellular membrane. This action results in the permeation or “pore formation” of the cell membrane, which allows introduction of bioactive substance(s) into the cells or exosomes. During the electroporation process, cells or exosomes are often suspended in a liquid media and then subjected to an electric field pulse. The medium may be electrolyte, nonelectrolyte, or a mixture of electrolytes and non-electrolytes.

The outcome of an electroporation process is largely controlled by the magnitude of the applied electrical field (EF) pulse and the duration of the pulse. Field strength is measured as the voltage delivered across an electrode gap and may be expressed as kV/cm. Field strength is critical to surpassing the electrical potential of the cell membrane to allow the temporary reversible permeation or pore formation to occur in the cell membrane, and the methods of the present disclosure are capable of subjecting the cells to a range of electric field strengths. Field strength is a function of several factors, including voltage magnitude of an applied electrical pulse, duration of the electrical pulse, and conductivity of the sample being electroporated.

The conductivity of the sample is a function of parameters comprising an ionic composition of electroporation buffer, concentration of an agent to be loaded, cell or exosome density, temperature, and pressure. Ionic strength of an electroporation buffer has a direct effect on the resistance of the sample, which in turn affects the pulse length or time constant of the pulse. The size and concentration of an agent will have an effect on the electrical parameters used to transfect the cell. Smaller molecules (for example, siRNA or miRNA) may need higher voltages with microsecond pulse lengths, while larger molecules (for example, DNA and proteins) may need lower voltages with longer pulse lengths. Cell or exosome density can be related to cell size. Generally, smaller cell or exosome sizes require higher voltages while larger cell or exosome sizes require lower voltages for successful cell membrane permeation.

Pulse duration, or pulse length, is the duration of time the sample is exposed to an electrical pulse and is typically measured as time in micro to milliseconds ranges. The pulse length works indirectly with the field strength to increase pore formation and therefore the uptake of target molecules. Generally, an increase in voltage should be followed by an incremental decrease in pulse length. Decreasing the voltage, the reverse is true. In addition to pulse duration, electrical pulses can also be characterized by pulse number, pulse width, pulse shape, pulse pattern, and pulse polarity. Thus, in some embodiments, the first and second electrical pulses further comprise characteristics selected from the group consisting of pulse number, width, shape, pattern, and polarity. Electroporation can be carried out as a single pulse or as multiple pulses as disclosed herein to achieve maximum transfection efficiencies. Pulse pattern can comprise a single pulse or multiple pulses, and a combined duration of the multiple pulses corresponds to the pulse duration. Pulse polarity can be positive or negative. Pulse width depends on the wave shape generated by a pulse generator of an electroporation system. Pulse shape, or wave form, generally falls into two categories, square wave or exponential decay wave. Square wave pulses rise quickly to a set voltage level and maintain this level during the duration of the set pulse length before quickly turning off. Exponential decay waves generate an electrical pulse by allowing a capacitor to completely discharge. A pulse is discharged into a sample, and the voltage rises rapidly to the peak voltage set then declines over time. The pulse width in an exponential decay wave system corresponds to the time constant and is characterized by the rate at which the pulsed energy or voltage is decayed to ⅓ the original set voltage. The time constant is modified by adjusting the resistance and capacitance values in an exponential decay, and the calculation for the time is T=RC, where T is time and R is resistance of a sample and C is capacitance of an electroporation system power supply. Thus, in some embodiments, the rate of exponential decay is a function of a resistance of the sample and the capacitance of a power supply used to effect electroporation.

The strength of the electric field applied to the suspension and the length of the pulse (the time that the electric field is applied to a cell suspension) varies according to the cell or exosome type. To create a pore in the outer membrane of a cell or exosome, the electric field must be applied for such a length of time and at such a voltage as to increase permeability of the membrane to allow the bioactive substance(s) to enter the cell or exosome. As long as the pulse magnitude is above a certain threshold level, an increase in either the magnitude or the duration of the pulse generally results in a greater accumulation of the bioactive substance(s) inside the cell or exosomes.

Each electrical pulse applied to a cell suspension can be characterized by a certain amount of energy, which is equal to the product of voltage on the electrodes, current through the buffer, and duration of the high voltage pulse. Electroporation parameters may be adjusted to optimize the strength of the applied electrical field and/or duration of exposure such that the pores formed in membranes by the electrical pulse reseal after a short period of time, during which bioactive substance(s) have a chance to enter into the cell or exosome.

Electroporation conditions may vary depending on the charge and size of the bioactive substance(s). Typical field strengths are in the range of 20 to 1000 V/cm or kV/cm, such as 20 to 100 V/cm or kV/cm. In some embodiments, field strengths are 0.01 to 10, 0.01 to 1, 0.1 to 10, 0.1 to 1, or 1 to 10 V/cm or kV/cm, or any value from 0.01 to 10 V/cm or kV/cm or range derivable therein. Field strength is a function of several factors, including voltage magnitude of an applied electrical pulse, duration of the electrical pulse, and conductivity of the sample being electroporated.

A voltage in the range of 150 mV or V to 250 mV or V, particularly a voltage of 200 mV or V may be used for loading exosomes with bioactive substance(s) according to the present disclosure. In some embodiments, the voltage magnitude of the electrical pulses is at most or at least about 0.001 to 10,000, 0.01 to 10,000, 0.1 to 10,000, 1 to 10,000, 1 to 9,000, 1 to 8,000, 1 to 7,000, 1 to 6,000, 1 to 5,000, 1 to 4,000, 1 to 3,000, 1 to 2,000, or 1 to 1,000 mV or V, or any value from 0.001 to 10,000 mV or V or range derivable therein. In some embodiments, the voltage magnitude of the electrical pulses is between 0.001 and 10,000, 0.01 and 10,000, 0.1 and 10,000, 1 and 10,000, 1 and 9,000, 1 and 8,000, 1 and 7,000, 1 and 6,000, 1 and 5,000, 1 and 4,000, 1 and 3,000, 1 and 2,000, or 1 and 1,000 mV or V, or any value from 0.001 to 10,000 mV or V or range derivable therein.

In some embodiments, the conductivity of the sample is a function of parameters comprising an ionic composition of electroporation buffer, concentration of an agent to be loaded into the cells, cell density, temperature, and pressure. In some embodiments, the conductivity of the sample is at most or at least about 0.01 Siemens/meter to 10 Siemens/meter, 0.01 Siemens/meter to 1 Siemens/meter, 0.1 Siemens/meter to 10 Siemens/meter, 0.1 Siemens/meter to 1 Siemens/meter, 1 Siemens/meter to 10 Siemens/meter, or any value from 0.01 Siemens/meter to 10 Siemens/meter or range derivable therein. In some embodiments, the conductivity of the sample is between 0.01 Siemens/meter and 10 Siemens/meter, 0.01 Siemens/meter and 1 Siemens/meter, 0.1 Siemens/meter and 10 Siemens/meter, 0.1 Siemens/meter and 1 Siemens/meter, 1 Siemens/meter and 10 Siemens/meter, or any value from 0.01 Siemens/meter to 10 Siemens/meter or range derivable therein. In some embodiments, the conductivity of the sample is between 1.0 and 3.0 Siemens/meter, any value from 1.0 Siemens/meter to 3.0 Siemens/meter, or any range or value derivable therein.

The ionic composition of a buffer used for electroporation can vary depending on the cell type. For example, highly conductive buffers such as PBS (Phosphate Buffered Saline <30 ohms) and HBSS (Hepes Buffer <30 ohms) or standard culture media, which may contain serum, may be used. Other buffers include hypoosmolar buffers in which cells absorb water shortly before an electrical pulse, which can result in cell swelling and can lower the optimal permeation voltage while ensuring the membrane is more easily permeable. Cells requiring the use of high resistance buffers (>3000 ohms) may require preparation and washing of the cells to remove excess salt ions to reduce the chance of arcing and sample loss. Ionic strength of an electroporation buffer has a direct effect on the resistance of the sample, which in turn affects the pulse length or time constant of the pulse. The volume of liquid in contact with an electrode also has significant effect on sample resistance for ionic solutions, and the resistance of the sample is inversely proportional to the volume of solution and pH. As volume increases, resistance decreases, which increases the probability of arcing and sample loss, while lowering the volume increases the resistance and decreases arc potential.

The size and concentration of an agent will have an effect on the electrical parameters used to transfect the cell. Smaller molecules (for example, siRNA or miRNA) may need higher voltages with microsecond pulse lengths, while larger molecules (for example, DNA and proteins) may need lower voltages with longer pulse lengths. The concentration of a bioactive substance may be, may be at least, may be at most, or may be from about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 20, 30, 50, 75, 100, 150, 200, 250, 300 to about 350, 400, 500, 1000, 1500, 2000, 3000, 4000, or 5000 μg/mL, mg/mL, or g/mL, or any value from 0.01 to 5000 μg/mL, mg/mL, or g/mL or range derivable therein. In further embodiments, the concentration of the bioactive substance is at least, at most, or exactly 1, 2, 3, 4, 5, 6, 7, 8, 9, 10, 15, 20, 25, 30, 35, 40, 45, 50, 55, 60, 65, 70, 75, 80, 85, 90, 95, 100, 150, 125, 150, 175, 200, 225, 250, 275, or 300 μg/mL, mg/mL, or g/mL, or any value from 1 to 300 g/mL, mg/mL, or g/mL or range derivable therein. In certain embodiments, the concentration of the bioactive substance is at least 1 μg/mL, mg/mL, or g/mL. In some embodiments, concentration of the bioactive substance is between 1 μg/mL and 200 μg/mL, such as between 5 μg/mL and 100 g/mL, any value from 5 μg/mL and 100 μg/mL, or any range derivable therein.

Cell density can be related to cell size. Generally, smaller cell sizes require higher voltages while larger cell sizes require lower voltages for successful cell membrane permeation. The temperature at which cells are maintained during electroporation can affect the efficiency of the electroporation. Samples pulsed at high voltage or exposed to multiple pulses and long pulse durations can cause sample heating, which can contribute to increased cell death and lower transfection efficiency. Maintaining the sample at a lower temperature can diminish the effects of overheating on cell viability and efficiency. In general, the standard pulse voltage used for cells at room temperature should be approximately doubled for electroporation at 4° C. in order to effectively permeate the cell membrane.

Pulse width depends on the wave shape generated by a pulse generator of an electroporation system. Pulse shape, or wave form, generally falls into two categories, square wave or exponential decay wave. Square wave pulses rise quickly to a set voltage level and maintain this level during the duration of the set pulse length before quickly turning off. In some embodiments, the pulse generator generates a square wave pulse, and pulse width can be inputted directly. Exponential decay waves generate an electrical pulse by allowing a capacitor to completely discharge. A pulse is discharged into a sample, and the voltage rises rapidly to the peak voltage set then declines over time. In some embodiments, the pulse generator generates an exponential decay wave pulse, and the pulse width is a function of a rate of exponential decay. The pulse width in an exponential decay wave system corresponds to the time constant and is characterized by the rate at which the pulsed energy or voltage is decayed to ⅓ the original set voltage. The time constant is modified by adjusting the resistance and capacitance values in an exponential decay, and the calculation for the time is T=RC, where T is time and R is resistance of a sample and C is capacitance of an electroporation system power supply. Thus, in some embodiments, the rate of exponential decay is a function of a resistance of the sample and the capacitance of a power supply used to effect electroporation.

The resistance of a sample can be at most or at least 1 ohm to 10000 ohms, 1 ohm to 9000 ohms, 1 ohm to 8000 ohms, 1 ohm to 7000 ohms, 1 ohm to 6000 ohms, 1 ohm to 5000 ohms, 1 ohm to 4000 ohms, 1 ohm to 3000 ohms, 1 ohm to 2000 ohms, 1 ohm to 1000 ohms, 1 ohm to 900 ohms, 1 ohm to 800 ohms, 1 ohm to 700 ohms, 1 ohm to 600 ohms, 1 ohm to 500 ohms, 1 ohm to 400 ohms, 1 ohm to 300 ohms, 1 ohm to 200 ohms, 1 ohm to 100 ohms, 1 ohm to 90 ohms, 1 ohm to 80 ohms, 1 ohm to 70 ohms, 1 ohm to 60 ohms, 1 ohm to 50 ohms, 1 ohm to 40 ohms, 1 ohm to 30 ohms, 1 ohm to 20 ohms, 1 ohm to 10 ohms, or any value from 1 ohm to 10000 ohms or range derivable therein. In some embodiments, the resistance of the sample is between 1 ohm and 10000 ohms, 1 ohm and 9000 ohms, 1 ohm and 8000 ohms, 1 ohm and 7000 ohms, 1 ohm and 6000 ohms, 1 ohm and 5000 ohms, 1 ohm and 4000 ohms, 1 ohm and 3000 ohms, 1 ohm and 2000 ohms, 1 ohm and 1000 ohms, 1 ohm and 900 ohms, 1 ohm and 800 ohms, 1 ohm and 700 ohms, 1 ohm and 600 ohms, 1 ohm and 500 ohms, 1 ohm and 400 ohms, 1 ohm and 300 ohms, 1 ohm and 200 ohms, 1 ohm and 100 ohms, 1 ohm and 90 ohms, 1 ohm and 80 ohms, 1 ohm and 70 ohms, 1 ohm and 60 ohms, 1 ohm and 50 ohms, 1 ohm and 40 ohms, 1 ohm and 30 ohms, 1 ohm and 20 ohms, 1 ohm and 10 ohms, or any value from 1 ohm to 10000 ohms or range derivable therein. In some embodiments, the resistance of the sample is between 1 ohm and 1000 ohms, any value from 1 ohm to 1000 ohms, or any range derivable therein.

The bioactive substances may be proteins and peptides (synthetic, natural, and mimetics, including antibodies or fragments thereof), oligonucleotides (anti-sense oligonucleotides, ribozymes, etc.), short nucleic acid sequences less than about 1000 nucleotides (e.g., double sense linear DNA, inhibitory RNA, siRNA, miRNA, anti-miRNA, shRNA, expression vectors, etc.), ribonucleoproteins, vectors, small molecules, lipids, carbohydrates, cytokines, hemobioactive substances, anti-cancer drugs, anti-inflammatory drugs, anti-fungal drugs, anti-viral drugs, anti-microbial drugs, thrombomodulating agents, immunomodulating agents, and the like.

In certain embodiments, the bioactive substance is miRNA, and the concentration of miRNA is between 1 μg/mL and 200 μg/mL, such as between 5 μg/mL and 100 μg/mL, any value from 5 μg/mL and 100 μg/mL, or any range derivable therein. In certain embodiments, the bioactive substance is siRNA, shRNA, and/or RNA, and the concentration of siRNA, shRNA, and/or RNA is between 1 μg/mL and 200 μg/mL, such as between 10 μg/mL and 50 μg/mL, any value from 10 μg/mL and 50 μg/mL, or any range derivable therein. In certain embodiments, the bioactive substance is DNA, the DNA is at least, at most, or about 1000 base pairs, and the concentration of DNA is between 1 μg/mL and 200 μg/mL, such as between 10 μg/mL and 100 g/mL, any value from 10 μg/mL and 100 μg/mL, or any range derivable therein.

In certain embodiments, the bioactive substance is protein, peptides, lipids, and/or drugs, the protein, peptides, lipids, and the concentration of protein, peptides, lipids, and/or drugs is between 1 μg/mL and 1000 mg/mL, such as between 100 μg/mL and 3 mg/mL, any value from 100 μg/mL and 3 mg/mL, or any range derivable therein, certain embodiments, the bioactive substance is protein, peptides, lipids, and/or drugs, the protein, peptides, lipids, and the concentration of protein, peptides, lipids, and/or drugs is 1 μg/mL or mg/mL, 10 μg/mL or mg/mL, 20 μg/mL or mg/mL, 960 μg/mL or mg/mL, 970 μg/mL or mg/mL, 980 μg/mL or mg/mL, 990 g/mL or mg/mL, 1000 μg/mL or mg/mL, or any range or value derivable therein.

In particular embodiments, the parameters for an electroporation pulse comprise a power between 100 to 240 VAC, with a frequency of between 50 to 60 Hz and a voltage of about 1500 V, with a limitation of 100 A. In some embodiments, all components for electroporation, including but not limited to buffers, exosomes and cuvettes or electrodes, should be kept at at least about 4° C. In some embodiments, the electroporation pulse is performed at at least about 25° C., and the electroporated exosomes are placed at at least about 4° C. following electroporation, for example, immediately following electroporation.

Electroporation is capable of achieving loading, or transfection, efficiencies of bioactive substance(s) into cells or exosomes of greater than 30%, greater than 40%, greater than 50%, greater than 60%, greater than 70%, greater than 80% or greater than 90% (or any range or value derivable therein). In some embodiments, a loading efficiency of bioactive substance(s) is at least 30%, at least 40%, at least 50%, at least 60%, at least 70%, at least 80%, or at least 90%. Transfection efficiency can be measured either by the percentage of the cells that express the product of the gene or the secretion level of the product expressed by the gene or by directly measuring concentration of the bioactive substance(s) in the exosomes using, for example, real-time quantitative PCR (RT-qPCR) or similar quantitative analyses.

### 3. Transfection

In particular embodiments of the disclosure, the MSCs and/or exosomes are loaded by use of a transfection reagent. Particular transfection reagents for use in accordance with the present disclosure include cationic lipids and/or liposomes.

The use of lipid formulations is contemplated for the introduction of the bioactive substance(s) into MSCs and/or exosomes. In another embodiment, the bioactive substance(s) may be associated with a lipid. The bioactive substance(s) associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid.

Lipid-, lipid/DNA-, lipid/expression vector-, or lipid/bioactive substance(s)-associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

In a certain embodiment, the bioactive substance(s) may be entrapped in a lipid complex such as, for example, a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). The amount of liposomes used may vary upon the nature of the liposome as well as the entity to be transfected, for example, about 5 to about 20 pg vector DNA per 1 to 10 million cells may be contemplated.

Liposome-mediated bioactive substance(s) delivery and expression of bioactive substance(s) in vitro has been very successful (Nicolau and Sene, 1982; Fraley et al, 1979; Nicolau e/al, 1987). The feasibility of liposome-mediated delivery and expression of bioactive substance(s) in cultured chick embryo, HeLa and hepatoma cells has also been demonstrated (Wong et al, 1980). In certain embodiments, a liposome may be complexed with a hemagglutinating virus (HVJ). This has been shown to facilitate fusion with the cell membrane and promote cell entry of liposome-encapsulated DNA (Kaneda et al, 1989). In other embodiments, a liposome may be complexed or employed in conjunction with nuclear non-histone chromosomal proteins (HMG-1) (Kato et al, 1991). In yet further embodiments, a liposome may be complexed or employed in conjunction with both HVJ and HMG-1. In other embodiments, a delivery vehicle may comprise a ligand and a liposome.

In various embodiments lipids suitable for use can be obtained from commercial sources. For example, lipofectamine can be obtained from Thermo Fisher Scientific, Waltham, Mass.; dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform can be used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al (1991) Glycobiology 5: 505-510). However, compositions that have different structures in solution than the normal vesicular structure are also encompassed. For example, the lipids may assume a micellar structure or merely exist as nonuniform aggregates of lipid molecules.

In specific embodiments, the exosomes are able to be loaded with any type of bioactive substance(s). Examples of suitable bioactive substance(s) include bioactive materials. Bioactive materials particularly suited to incorporation into exosomes include, but are not limited to, therapeutic and prophylactic agents. Examples of bioactive materials include, but are not limited to, proteins and peptides (synthetic, natural, and mimetics, including antibodies or fragments thereof), oligonucleotides (anti-sense oligonucleotides, ribozymes, etc.), short nucleic acid sequences less than about 1000 nucleotides (e.g., double sense linear DNA, inhibitory RNA, siRNA, miRNA, anti-miRNA, shRNA, expression vectors, etc.), ribonucleoproteins, vectors, small molecules, lipids, carbohydrates, cytokines, hemobioactive substances, anti-cancer drugs, anti-inflammatory drugs, anti-fungal drugs, antiviral drugs, anti-microbial drugs, thrombomodulating agents, immunomodulating agents, and the like.

It is to be understood that other bioactive substance(s) can also be introduced into the exosomes. These bioactive substances of interest include, but are not limited to, smooth muscle inhibitors, anti-infective bioactive substances (e.g., antibiotics, antifungal agents, antibacterial agents, antiviral agents), chemotherapeutic/antineoplastic agents, and the like. The bioactive substance(s) may be cancer bioactive substances, bioactive substances for auto- or alloimmune disease, bioactive substances for microbial infection, bioactive substances for heart disease, bioactive substances for lung disease, bioactive substances for liver disease, bioactive substances for kidney disease, bioactive substances for neurological disease, or a combination thereof. For cancer bioactive substances, the bioactive substance(s) may be, for example, a drug, small molecule, antibody, inhibitory RNA targeting an oncogene, tumor suppressor protein, or a combination or mixture thereof.

In some embodiments, the exosomes comprise one or more short DNA sequences and/or one or more short RNA sequences. The one or more short RNA sequences may comprise inhibitory RNA, including miRNA, anti-miRNA, siRNA, shRNA, Morpholino oligomers, or a combination or mixture thereof. A microRNA (“miRNA” or “miR”) refers to a small single-stranded non-coding RNA molecule that functions in RNA silencing and post-transcriptional regulation of gene expression by base-pairing with complementary sequences within mRNA molecules. Upon base-pairing between the miRNA and complementary mRNA molecule, the mRNA molecule is silenced by either cleavage of the mRNA strand into two pieces, destabilization of the mRNA through shortening of its poly(A) tail, and/or less efficient translation of the mRNA into proteins by ribosomes. Anti-miRNA (also known as “anti-miRNA oligonucleotide” or “AMO”) refers to synthetically designed molecules used to neutralize miRNA function in cells. By controlling the miRNA that regulate mRNAs in cells, AMOs can be used as further regulation through, for example, a steric blocking mechanism as well as hybridization to miRNA. These interactions between miRNA and AMOs can be therapeutic in disorders in which miRNA over/under expression occurs or aberrations in miRNA lead to coding issues. Small interfering RNA (“siRNA” or “short interfering RNA” or “silencing RNA” refers to a class of double-stranded RNA non-coding RNA molecules that operate in sequence-specific suppression of gene expression. siRNA interfere with the expression of specific genes with complementary nucleotide sequences by degrading mRNA after transcription, thereby preventing translation of the mRNA into amino acids and then proteins. siRNA may be introduced into cells using an expression vector in which the siRNA sequence is modified to introduce a short loop between the two strands. The resulting transcript is a short hairpin RNA (“shRNA” or “short hairpin RNA”), which can be processed into a functional siRNA by Dicer, an enzyme that cleaves double-stranded RNA into siRNA (and pre-microRNA into microRNA). Morpholino oligomers (“morpholino” or “phosphorodiamidate morpholino oligomer” or “PMO”) refer to oligomer molecules containing DNA bases attached to a backbone of methylenemorpholine rings linked through phosphorodiamidate groups. Morpholinos sterically block access of other molecules to small specific sequences of the base-pairing surfaces of RNA, thereby modifying gene expression. For example, Morpholinos can modify pre-mRNA splicing, block translation by interfering with progression of the ribosomal initiation complex from the 5′ cap to the start codon, or block other functional sites on RNA (i.e., blocking miRNA activity and maturation, blocking ribozyme activity, etc.) depending on the Morpholino's base sequence.

In some embodiments, the exosomes comprise one or more antibodies or antibody fragments. The term “antibody” as referred to herein includes whole antibodies and any antigen binding fragment (i.e., “antigen-binding portion”) or single chains thereof. An antibody refers to a glycoprotein comprising at least two heavy (H) chains and two light (L) chains interconnected by disulfide bonds, or an antigen binding portion thereof. Each heavy chain is comprised of a heavy chain variable region (abbreviated herein as VH) and a heavy chain constant region. Each light chain is comprised of a light chain variable region (abbreviated herein as VL) and a light chain constant region. The variable regions of the heavy and light chains contain a binding domain that interacts with an antigen. The VH and VL regions can be further subdivided into regions of hypervariability, termed complementarity determining regions (CDR), interspersed with regions that are more conserved, termed framework regions (FR). An antibody of use in the invention may be a monoclonal antibody or a polyclonal antibody and will preferably be a monoclonal antibody.

An antibody of use in the invention may be a chimeric antibody, a CDR-grafted antibody, a nanobody, a human or humanized antibody or an antigen binding portion of any thereof. For the production of both monoclonal and polyclonal antibodies, the experimental animal is typically a non-human mammal such as a goat, rabbit, rat or mouse but may also be raised in other species such as camelids.

The term “antigen-binding portion” of an antibody refers to one or more fragments of an antibody that retain the ability to specifically bind to an antigen. It has been shown that the antigen-binding function of an antibody can be performed by fragments of a full-length antibody. Examples of binding fragments encompassed within the term “antigen binding portion” of an antibody include a Fab fragment, a F(ab′)2 fragment, a Fab′ fragment, a Fd fragment, a Fv fragment, a dAb fragment, and an isolated complementarity determining region (CDR). Single chain antibodies such as scFv antibodies are also intended to be encompassed within the term “antigen-binding portion” of an antibody. These antibody fragments may be obtained using conventional techniques known to those of skill in the art, and the fragments may be screened for utility in the same manner as intact antibodies. An antibody of use in the invention may be a human antibody or a humanized antibody.

In some embodiments, the exosomes are loaded with one or more cancer drugs, including one or more chemotherapies. A wide variety of chemotherapeutic substances may be used in accordance with the present embodiments. The term “chemotherapy” refers to the use of drugs to treat cancer. A “chemotherapy” or “chemotherapeutic substance” is used to connote a compound or composition that is administered in the treatment of cancer. These bioactive substances or drugs are categorized by their mode of activity within a cell, for example, whether and at what stage they affect the cell cycle. Alternatively, a bioactive substance may be characterized based on its ability to directly cross-link DNA, to intercalate into DNA, or to induce chromosomal and mitotic aberrations by affecting nucleic acid synthesis.

Examples of chemotherapies include alkylating agents, such as thiotepa, procarbazine, and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; cally statin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancrati statin; a sarcodictyin; spongistatin; nitrogen mustards, such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as plicomycin and the enediyne antibiotics (e.g., calicheamicin, especially calicheamicin gammall and calicheamicin omegall); dynemicin, including dynemicin A; bisphosphonates, such as clodronate; an esperamicin; as well as neocarzinostatin chromophore and related chromoprotein enediyne antibiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), adriamycin, epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, gemcitabine, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., taxol, paclitaxel, and docetaxel; 6-thioguanine; mercaptopurine; platinum coordination complexes, such as transplatinum, cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; protease inhibitors, like bortezomib; kinase inhibitors, like palbociclib, ibrutinib, dasatinib, pp2, pazopanib, and gefitinib; checkpoint inhibitors, like nivolumab, pembrolizumab, and ipilimumab; colony stimulating factors, like pegfilgrastim and filgrastim; monoclonal antibodies, like bevacizumab, trastuzumab, and rituximab; immunomodulatory agents, like lenalidomide; navelbine; farnesyl-protein transferase inhibitors; pharmaceutically acceptable salts, acids, or derivatives of any of the above; and combinations thereof.

In certain embodiments, the exosomes are loaded with one or more antimicrobial agents. An antimicrobial agent may be a natural or synthetic substance that kills or inhibits the growth of microorganisms or pathogens, such as bacteria, fungi, algae, or viruses. The antimicrobial agents may be an antibiotic, antifungal, antiviral, and so forth.

Examples of antibiotics include but are not limited to aminoglycosides, ansamycins, carbacephem, carbapenems, cephalosporins (first, second, third, fourth, or fifth generation), glycopeptides, linocsamides, lipopeptides, macrolides, monobactams, nitrofurans, oxazolidinones, penicillins, polypeptides, quinolones/fluoroquinolones, sulfonamides, tetracyclines, clofazimine, dapsone, capreomycin, cycloserine, ethambutol, ethionamide, isoniazid, pyrazinamide, rifampicin, rifabutin, rifapentine, streptomycin, arsphenamine, chloramphenicol, fosfomycin, fusidic acid, metronidazole, mupirocin, platensimycin, quinupristin/dalfopristin, thiamphenicol, tigecycline, tinidazole, trimethoprim, and combinations thereof. Aminoglycosides can include, but are not limited to: Amikacin, Gentamicin, Kanamycin, Neomycin, Netilmicin, Tobramycin, Paromomycin, Streptomycin, and Spectinomycin. Ansamycins can include but are not limited to: Geldanamycin, Herbimycin, and Rifaximin. Carbacephem can include but is not limited to Loracarbef. Carbapenems can include but are not limited to Ertapenem, Doripenem, Imipenem/Cilastatimn, and Meropenem. Cephalosporins can include but are not limited to: Cefadroxil, Cefazolin, Cephradine, Cephapirin, Cephalothin, Cefalexin, Cefaclor, Cefoxitin, Cefotetan, Cefotan, Cefamandole, Cefmetazole, Cefonicid, Loracarbef, Cefprozil, Cefzil, Cefuroxime, Cefixime, Cefdinir, Cefditoren, Cefoperazone, Cefotaxime, Cefpodoxime, Ceftazidime, Ceftibuten, Ceftizoxime, Moxalactam, Ceftriaxone, Cefepime, Ceftaroline fosamil, and Ceftobiprole. Glycopeptides can include but are not limited to: Teicoplanin, Vancomycin, Telavancin, Dalbavancin, and Oritavancin. Lincosamides can include but are not limited to Clindamycin and Lincomycin. Lipopeptides can include but are not limited to Daptomycin. Macrolides can include but are not limited to: Azithromycin, Clarithromycin, Erythromycin, Roxithromycin, Telithromycin, Spiramycin, and Fidaxomicin. Monobactams can include but are not limited to Aztreonam. Nitrofurans can include but are not limited to: Furazolidone and Nitrofurantoin. Oxazolidinones can include but are not limited to: Linezolid, Posizolid, Radezolid, and Torezolid. Penicillins can include but are not limited to: Amoxicillin, Ampicillin, Azlocillin, Dicloxacillin, Flucloxxacillin, Mezlocillin, Methicillin, Nafcillin, Oxacillin, Penicillin G, Penicillin V, Piperacillin, Temocillin, Ticarcillin, Amoxicillin/clavulanate, Ampicillin/sulbactam, Piperacillin/tazobactam, and Ticarcillin/clavulanate. Polypeptides can include but are not limited to: Bacitracin, Colistin, and Polymyxin B. Quinolones/fluoroquinolones can include but are not limited to: Ciprofloxacin, Enoxacin, Gatifloxacin, Gemifloxacin, Levofloxacin, Lomefloxacin, Moxifloxacin, Nadifloxacin, Nalidixic acid, Norfloxacin, Ofloxacin, Trovafloxacin, Grepafloxacin, Sparfloxacin, and Temafloxacin. Sulfonamides can include but are not limited to: Mafenide, Sulfacetamide, Sulfadiazine, Silver sulfadiazine, Sulfadimethoxine, Sulfamethizole, Sulfamethoxazole, Sulfanilimide, Sulfasalazine, Sulfisoxazole, Trimethoprim-Sulfamethoxazole, and Sulfoamidochrysoidine. Tetracyclines can include but are not limited to: Demeclocycline, Doxycydine, Metacycline, Minocycline, Oxytetracycline, and Tetracycline. In some embodiments, the antibiotic is a macrolide. In some embodiment, the antibiotic is azithromycin.

Examples of antibiotics also include but are not limited to antimicrobial proteins or peptides. The antimicrobial proteins or peptides can be of any class, including but not limited to the following classes: anionic peptides (e.g., dermicidin), linear cationic a-helical peptides (e.g., LL37), cationic peptides enriched for proline, arginine, phenylalanine, glycine, or tryptophan, anionic and cationic peptides that contain cysteine and form disulfide bonds (e.g., defensins), and combinations thereof. Defensins can include but are not limited to trans-defensins, cis-defensins, and related defensin-like proteins. Trans-defensins include but are not limited to a-defensins and b-defensins.

Examples of antibiotics also include but are not limited to anti-mycobacterials, including, but not limited to, isoniazid, rifampin, streptomycin, rifabutin, ethambutol, pyrazinamide, ethionamide, aminosalicylic, and cycloserine. Examples of antivirals include but are not limited to anti-herpes agents such as acyclovir, famciclovir, foscamet, ganciclovir, acyclovir, idoxuridine, sorivudine, trifluridine, valacyclovir and vidarabine; anti-retroviral agents such as ritonavir, didanosine, stavudine, zalcitabine, tenovovir and zidovudine; and other antiviral agents such as, but not limited to, amantadine, interferon-alpha, ribavirin, rimantadine, and combinations thereof.

Examples of antifungals include but are not limited to polyene antifungals (e.g., amphotericin B, nystatin, natamycin, and the like), flucytosine, imidazoles (e.g., n-ticonazole, clotrimazole, econazole, ketoconazole, and the like), triazoles (e.g., itraconazole, fluconazole, and the like), griseofulvin, terconazole, butoconazole ciclopirax, ciclopirox olamine, haloprogin, tolnaftate, naftifme, terbinafme, any other antifungal that can be lipid encapsulated or complexed, and combinations thereof.

In some embodiments, the exosomes are loaded with one or more bioactive substances for the treatment of an auto- or alloimmune disease. Examples of auto- or alloimmune disease therapies include but are not limited to anti-microbial agents (for example, antibiotics, antiviral agents and anti-fungal agents), anti-tumor agents (for example, fluorouracil, methotrexate, paclitaxel, fludarabine, etoposide, doxorubicin, or vincristine), immune-depleting agents (for example, fludarabine, etoposide, doxorubicin, or vincristine), immunosuppressive agents (for example, azathioprine, or glucocorticoids, such as dexamethasone or prednisone), anti-inflammatory agents (for example, glucocorticoids such as hydrocortisone, dexamethasone or prednisone, or non-steroidal anti-inflammatory agents such as acetylsalicylic acid, ibuprofen or naproxen sodium), cytokines (for example, interleukin-10 or transforming growth factor-beta), hormones (for example, estrogen), or a vaccine. In addition, immunosuppressive or tolerogenic agents including but not limited to calcineurin inhibitors (e.g., cyclosporin and tacrolimus); mTOR inhibitors (e.g., Rapamycin); mycophenolate mofetil, antibodies (e.g., recognizing CD3, CD4, CD40, CD154, CD45, IVIG, or B cells); chemotherapeutic substances (e.g., Methotrexate, Treosulfan, Busulfan); irradiation; or chemokines, interleukins, or their inhibitors (e.g., BAFF, IL-2, anti-IL-2R, IL-4, JAK kinase inhibitors) can be administered.

In alternative embodiments, the exosomes are not loaded with a therapeutic drug but instead are loaded with one or more gene-modifying components, such as that comprise a CRISPR-Cas system, including a specific guide RNA and an endonuclease. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus. The CRISPR/Cas nuclease or CRISPR/Cas nuclease system can include a non-coding RNA molecule (guide) RNA, which sequence-specifically binds to DNA, and a Cas protein (e.g., Cas9), with nuclease functionality (e.g., two nuclease domains).

In some embodiments, a Cas nuclease and gRNA (including a fusion of crRNA specific for the target sequence and fixed tracrRNA) are introduced into the cell. In general, a guide sequence is any polynucleotide sequence having sufficient complementarity with a target polynucleotide sequence to hybridize with the target sequence and direct sequence-specific binding of the CRISPR complex to the target sequence. Typically, “target sequence” generally refers to a sequence to which a guide sequence is designed to have complementarity, where hybridization between the target sequence and a guide sequence promotes the formation of a CRISPR complex. The CRISPR system can induce double stranded breaks (DSBs) at the target site, followed by disruptions or alterations as discussed herein. In other embodiments, Cas9 variants, deemed “nickases,” are used to nick a single strand at the target site. In other embodiments, catalytically inactive Cas9 is fused to a heterologous effector domain such as a transcriptional repressor or activator, to affect gene expression. The target sequence may comprise any polynucleotide, such as DNA or RNA polynucleotides. The target sequence may be located in the nucleus or cytoplasm of the cell, such as within an organelle of the cell.

In some embodiments, exosomes loaded with one or more vectors can be introduced into cells to drive expression of one or more elements of the CRISPR system such that expression of the elements of the CRISPR system direct formation of the CRISPR complex at one or more target sites. Components can also be delivered to cells via exosomes as proteins and/or RNA. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. The vector may comprise one or more insertion sites, such as a restriction endonuclease recognition sequence (also referred to as a “cloning site”). In some embodiments, one or more insertion sites are located upstream and/or downstream of one or more sequence elements of one or more vectors. When multiple different guide sequences are used, a single expression construct may be used to target CRISPR activity to multiple different, corresponding target sequences within a cell. A vector may comprise a regulatory element operably linked to an enzyme-coding sequence encoding the CRISPR enzyme, such as a Cas protein.

### V. MSC-Exos Methods of Use

In particular embodiments, exosomes are useful for the treatment of one or more medical conditions. The exosomes may be used for the systemic or local delivery of therapeutic compounds. The disclosure encompasses methods for delivering bioactive substances of interest using exosomes as a delivery vehicle. The present disclosure also includes methods of treating a patient in need of one or more bioactive substances comprising administering to the patient an effective amount of exosomes containing the bioactive substance(s).

The exosomes of the disclosure may or may not be utilized directly after production. In some cases they are stored for later purpose. In any event, they may be utilized in therapeutic or preventative applications for a mammalian subject (human, dog, cat, horse, etc.) such as a patient. The individual may be in need of exosome-based therapy for a medical condition of any kind, including cancer, infections of any kind, any immune disorder, any tissue injury, any skin disorder, any wounds, any trauma, and/or any burns, as examples. Methods may be employed with respect to individuals who have tested positive for a medical condition, who have one or more symptoms of a medical condition, or who are deemed to be at risk for developing such a condition.

Individuals treated with the present exosome-based therapy may or may not have been treated for the particular medical condition prior to receiving the exosome-based therapy. In some embodiments, the patient has received at least 1, 2, 3, 4, 5, 6, 7, 8, or more prior treatments for a medical condition. The prior treatments may include a treatment or therapy described herein. In some embodiments, the prior treatments comprise conventional chemotherapies, conventional radiotherapy, conventional antiviral therapies, conventional antiseptic and antibacterial therapies, conventional immunosuppressive therapies, conventional anti-inflammatory therapies, conventional bum treatment therapies, and the like. In some embodiments, the patient had received the prior therapy within 10, 20, 30, 40, 50, 60, 70, 80, or 90 days or hours of administration of the current compositions and exosomes of the disclosure. In some embodiments, the patient is one that has undergone prior therapy and has failed the prior treatment either because the prior treatment was not effective or because the prior treatment was deemed too toxic.

Exosomes loaded with one or more bioactive substances as contemplated herein, and/or pharmaceutical compositions comprising the same, can be administered either alone or in any combination, and in at least some embodiments, together with a pharmaceutically acceptable carrier or excipient, and can be used for the prevention, treatment, or amelioration of cancer, immune disorders, heart disease, lung disease, microbial infections, tissue injuries, skin disorders, wounds, trauma, and/or burns of any kind. Exosomes loaded with one or more bioactive substances as contemplated herein, and/or pharmaceutical compositions comprising the same, can also be used for the mitigation of chemo- and radiotherapy-induced CNS toxicity, and the treatment of other chemotherapy or radiation-induced vital organ toxicities involving the heart, lung, kidney, gastrointestinal tract where regenerative or reparative properties are often needed.

Exosomes loaded with one or more bioactive substances as contemplated herein, and/or pharmaceutical compositions comprising the same, can also be used for the mitigation of chemo- and radiotherapy-induced CNS toxicity, and the treatment of other chemotherapy or radiation-induced vital organ toxicities involving the heart, lung, kidney, gastrointestinal tract where regenerative or reparative properties are often needed.

Aspects of the disclosure include methods for treating, reversing, or ameliorating cognitive impairment in response to or providing neuroprotection against chemo- and radiotherapy-induced central nervous system (CNS) toxicity. In specific embodiments, exosomes derived from umbilical cord tissue-derived MSCs (UC-Exos) are useful for treating or reversing cognitive impairment in response to or providing neuroprotection against chemo- and radiotherapy-induced central nervous system (CNS) toxicity. Methods and compositions of the disclosure allow for generation of a large scale of activated exosomes from UC-Exos, carrying bioactive substance(s), including at least miR, anti-miR, siRNA, and therapeutic drugs for treating or reversing cognitive impairment in response to or providing neuroprotection against chemo- and radiotherapy-induced central nervous system (CNS) toxicity.

In certain embodiments, the exosomes (e.g., UC-Exos) are utilized for individuals in need of regeneration and/or reparation of tissue for any reason. The tissue in need of regeneration and/or reparation may be of any kind, but in specific embodiments the tissue is soft tissue (e.g., fat, fibrous tissue (e.g., tendons and/or ligaments), muscle (e.g., smooth muscle, skeletal muscle, and/or cardiac muscle), synovial tissue, blood vessels, lymph vessels, and/or nerves), brain, lung, spleen, liver, heart, kidney, pancreas, intestine, testis, or bone. For example, the individual may be in need of regeneration and/or reparation of heart, lung, kidney, and/or gastrointestinal tract tissue due to chemotherapy or radiation-induced vital organ toxicities. The individual may be in need of regeneration and/or reparation of soft tissue (e.g., fat, fibrous tissue (e.g., tendons and/or ligaments)), muscle (e.g., smooth muscle, skeletal muscle, and/or cardiac muscle), synovial tissue, blood vessels, lymph vessels, and/or nerves) due to inflammation, trauma (e.g., contusions, sprains, tendonitis, bursitis, stress injuries, strains), burns (e.g., thermal burns, chemical burns, electric burns, frostbite) or a combination thereof. In some embodiments, the tissue is in need of regeneration or repair due to toxicity due to burns (e.g., thermal burns, chemical burns, electric burns, frostbite) or trauma (e.g., contusions, sprains, tendonitis, bursitis, stress injuries, strains) and/or due to toxicity due to a prior treatment for burns (e.g., thermal burns, chemical burns, electric burns, frostbite) or trauma (e.g., contusions, sprains, tendonitis, bursitis, stress injuries, strains). In particular embodiments, the exosomes produced by methods encompassed herein are useful as regenerative and/or reparative therapies to target soft tissues and organs including brain, lung, spleen, liver, heart, kidney, pancreas, intestine, testis, and bone, as examples of target tissues. The exosomes in such cases are therapeutic at least in part because they are suitable to migrate in the individual.

In certain embodiments, the exosomes (e.g., UC-Exos) are utilized for individuals in need of regeneration and/or reparation of skin for any reason. For example, the individual may be in need of regeneration and/or reparation of skin due to chemotherapy or radiation-induced vital organ toxicities. The individual may be in in need of regeneration and/or reparation of skin due to toxicity due to burns (e.g., thermal burns, chemical burns, electric burns, frostbite) or trauma (e.g., cutaneous wound damage, contusions, cuts, lacerations, gashes, tears, punctures, scrapes, abrasions, scratches, bites, stings, bruises, pressure injuries, crush injuries, incisions) and/or due to toxicity due to a prior treatment administered for burns (e.g., thermal burns, chemical burns, electric burns, frostbite) or trauma (e.g., cutaneous wound damage, contusions, cuts, lacerations, gashes, tears, punctures, scrapes, abrasions, scratches, bites, stings, bruises, pressure injuries, crush injuries, incisions). The individual may be in need of regeneration and/or reparation of skin due to a skin disorder. Non-limiting examples of skin disorders include inflammation, aging, skin cancer, acne, cold sores, blisters, seromas, hematomas, ulcers, carbuncles, warts, psoriasis, eczema, cellulitis, lupus, actinic keratosis, keratosis pilaris, shingles, hives, melasma, impetigo, sunburn, dermatitis, rosacea, thermal burns, chemical burns, electric burns, frostbite, cutaneous wound damage, contusions, cuts, lacerations, gashes, tears, punctures, scrapes, abrasions, scratches, bites, stings, bruises, pressure injuries, crush injuries, incisions, or a combination thereof.

In certain embodiments, the exosomes (e.g., UC-Exos) are utilized for individuals in need of wound healing (e.g., wound repair) for any reason. For example, the individual may be in need of regeneration and/or reparation of wounded skin or tissue due to chemotherapy or radiation-induced vital organ toxicities. The individual may be in need of regeneration and/or reparation of wounded skin or tissue due to toxicity due to burns (e.g., thermal burns, chemical burns, electric burns, frostbite) or trauma (e.g., sprains, tendonitis, bursitis, stress injuries, strains, cutaneous wound damage, contusions, cuts, lacerations, gashes, tears, punctures, scrapes, abrasions, scratches, bites, stings, bruises, pressure injuries, crush injuries, incisions) and/or toxicity due to a prior treatment for burns (e.g., thermal burns, chemical burns, electric burns, frostbite) or trauma (e.g., sprains, tendonitis, bursitis, stress injuries, strains, cutaneous wound damage, contusions, cuts, lacerations, gashes, tears, punctures, scrapes, abrasions, scratches, bites, stings, bruises, pressure injuries, crush injuries, incisions). The individual may be in need of regeneration and/or reparation of wounded skin or tissue due to inflammation, aging, skin cancer, acne, cold sores, blisters, seromas, hematomas, ulcers, carbuncles, warts, psoriasis, eczema, cellulitis, lupus, actinic keratosis, keratosis pilaris, shingles, hives, melasma, impetigo, sunburn, dermatitis, rosacea, thermal burns, chemical burns, electric burns, frostbite, cutaneous wound damage, contusions, cuts, lacerations, gashes, tears, punctures, scrapes, abrasions, scratches, bites, stings, bruises, pressure injuries, crush injuries, incisions, sprains, tendonitis, bursitis, stress injuries, strains, or a combination thereof.

Aspects of the disclosure include methods for treatment of cancer. In some cases, the exosomes are useful for one or more cancers. In specific embodiments, exosomes derived from umbilical cord tissue-derived MSCs (UC-Exos) are useful for the treatment of cancer and for the systemic delivery of therapeutic compounds for the cancer. Methods and compositions of the disclosure allow for generation of a large scale of activated exosomes from UC-Exos, carrying bioactive substance(s), including at least miR, anti-miR, siRNA, and therapeutic drugs for the treatment of cancer.

In some embodiments, the present invention includes a composition for delivering target specific exosomes to the cytoplasm of a tumor cell, wherein the exosomes modulate angiogenesis. In embodiments, the composition comprises an exosome, a bioactive substance, and/or a plasmid. In other embodiments, the exosome is isolated from autologous cells of a subject, from a cell line, from a primary cell culture, and/or from a mesenchymal stem cell. In other embodiments, the at least one plasmid is an RNA plasmid, a DNA plasmid, or any combination thereof.

Cancers for which the present exosomes are useful include any malignant cell type, such as those found in a solid tumor or a hematological tumor. In cases wherein the individual has cancer, the cancer may be primary, metastatic, resistant to therapy, and so forth. In specific cases, the present therapy is useful for individuals with cancers that have been clinically indicated to be subject to immune cell regulation, including multiple types of solid tumors (melanoma, colon, lung, breast, and head and neck cancers), for example. Exemplary solid tumors can include, but are not limited to, a tumor of an organ selected from the group consisting of pancreas, colon, cecum, stomach, brain, head, neck, ovary, kidney, larynx, sarcoma, lung, bladder, melanoma, prostate, and breast. Exemplary hematological tumors include tumors of the bone marrow, T or B cell malignancies, leukemias, lymphomas, blastomas, myelomas, and the like. Further examples of cancers that may be treated using the methods provided herein include, but are not limited to, glioblastoma, lung cancer (including small-cell lung cancer, non-small cell lung cancer, adenocarcinoma of the lung, and squamous carcinoma of the lung), cancer of the peritoneum, gastric or stomach cancer (including gastrointestinal cancer and gastrointestinal stromal cancer), pancreatic cancer, cervical cancer, ovarian cancer, liver cancer, bladder cancer, breast cancer, colon cancer, colorectal cancer, endometrial or uterine carcinoma, salivary gland carcinoma, kidney or renal cancer, prostate cancer, vulval cancer, thyroid cancer, various types of head and neck cancer, and melanoma.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; non-encapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; lentigo malignant melanoma; acral lentiginous melanomas; nodular melanomas; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; Kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; paragranuloma; malignant lymphoma, small lymphocytic; malignant lymphoma, large cell, diffuse; malignant lymphoma, follicular; mycosis fungoides; other specified non-Hodgkin's lymphomas; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; hairy cell leukemia; chronic lymphocytic leukemia (CLL); acute lymphoblastic leukemia (ALL); acute myeloid leukemia (AML); and chronic myeloblastic leukemia.

In some embodiments, cancers for which the present exosomes are useful is glioblastoma multiforme (GBM). Adult glioblastoma is notoriously recalcitrant to most therapies, not only because its molecular, cellular and immune biology are unique compared with other cancers, but also because of the formidable delivery challenges imposed by the blood brain/blood tumor barriers (BBB/BTB). Consequently, there is an urgent need to identify anticancer therapeutics that specifically target GBMs, and to elucidate strategies for delivering these new agents across the BBB/BTB. In some cases, exosomes home efficiently to human gliomas, overcoming the BBB/BTB.

In some embodiments, exosomes used to treat GBM are loaded with the anti-GMB miRNA miR-124. Validation studies proved that miR-124 is highly efficacious against all subtypes of glioma stem cells, functioning by down-regulating GBM-relevant targets, particularly FOXA2, and leading to apoptotic cell death. MiR-124 also enhances T-cell responses by inhibiting STAT-3, a known mediator of immune suppression in GBM, further supporting its therapeutic potential. Recent work has also shown that miR-124 reverses neurodegeneration after brain injury, rendering miR-124 one of the first anti-glioma agents that may also mitigate neuro-toxicity.

Aspects of the disclosure include methods for treatment of immune disorders. In some cases, the exosomes are useful for one or more immune disorders. In specific embodiments, exosomes derived from umbilical cord tissue-derived MSCs (UC-Exos) are useful for the treatment of immune disorders and for the systemic delivery of therapeutic compounds for the immune disorders. Methods and compositions of the disclosure allow for generation of a large scale of activated exosomes from UC-Exos, carrying bioactive substance(s), including at least miR, anti-miR, siRNA, and therapeutic drugs, for the treatment of immune disorders.

Immune disorders for which the present exosomes are useful include autoimmune or inflammatory disorders. Non-limiting examples of autoimmune or inflammatory disorders include: alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac spate-dermatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, Crohn's disease, discoid lupus, essential mixed cryoglobulinemia, fibromyalgia-fibromyositis, glomerulonephritis, Graves' disease, Guillain-Barre, Hashimoto's thyroiditis, idiopathic pulmonary fibrosis, idiopathic thrombocytopenia purpura (ITP), IgA neuropathy, juvenile arthritis, lichen planus, lupus erthematosus, Meniere's disease, mixed connective tissue disease, multiple sclerosis, type 1 or immune-mediated diabetes mellitus, myasthenia gravis, nephrotic syndrome (such as minimal change disease, focal glomerulosclerosis, or mebranous nephropathy), pemphigus vulgaris, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynaud's phenomenon, Reiter's syndrome, Rheumatoid arthritis, sarcoidosis, scleroderma, Sjogren's syndrome, stiff-man syndrome, systemic lupus erythematosus, lupus erythematosus, ulcerative colitis, uveitis, vasculitides (such as polyarteritis nodosa, takayasu arteritis, temporal arteritis/giant cell arteritis, or dermatitis herpetiformis vasculitis), vitiligo, graft versus host disease (GVHD), and Wegener's granulomatosis. Thus, some examples of an autoimmune disease that can be treated using the methods disclosed herein include, but are not limited to, multiple sclerosis, rheumatoid arthritis, systemic lupus erythematosis, type I diabetes mellitus, Crohn's disease; ulcerative colitis, myasthenia gravis, glomerulonephritis, ankylosing spondylitis, vasculitis, or psoriasis. The subject can also have an allergic disorder such as asthma.

Aspects of the disclosure include methods for treatment of heart disease of any kind, including at least coronary artery disease, heart failure, cardiomyopathy, valvular heart disease, arrhythmia, genetic defects of the heart, and so forth. Aspects of the disclosure include methods for treatment of lung disease, such as pulmonary hypertension, asthma, bronchopulmonary dysplasia (BPD), allergy, cystic fibrosis, Chronic Obstructive Pulmonary Disease, idiopathic pulmonary fibrosis, acute respiratory distress syndrome (ARDS), pneumonia, pleural effusion, and so forth.

Aspects of the disclosure include methods for treatment of a microbial infection of any kind, including a pathogenic infection. The infection may be bacterial, viral, fungal, or protozoan. Examples of bacteria include, but are not limited to, Actinomyces, Bacillus, Bacteroides, Bordetella, Bartonella, Borrelia, Brucella, Campylobacter, Capnocytophaga, Chlamydia, Corynebacterium, Coxiella, Dermatophilus, Enterococcus, Ehrlichia, Escherichia, Francisella, Fusobacterium, Haemobartonella, Haemophilus, Helicobacter, Klebsiella, L-form bacteria, Leptospira, Listeria, Mycobacteria, Mycoplasma, Neisseria, Neorickettsia, Nocardia, Pasteurella, Peptococcus, Peptostreptococcus, Pneumococcus, Proteus, Pseudomonas, Rickettsia, Rochalimaea polypeptides, Salmonella, Shigella, Staphylococcus, group A streptococcus, group B streptococcus, Treponema, and Yersinia. Examples of fungi include, but are not limited to, Absidia, Acremonium, Alternaria, Aspergillus, Basidiobolus, Bipolaris, Blastomyces, Candida, Coccidioides, Conidiobolus, Cryptococcus, Curvalaria, Epidermophyton, Exophiala, Geotrichum, Histoplasma, Madurella, Malassezia, Microsporum, Moniliella, Mortierella, Mucor, Paecilomyces, Penicillium, Phialemonium, Phialophora, Prototheca, Pseudallescheria, Pseudomicrodochium, Pythium, Rhinosporidium, Rhizopus, Scolecobasidium, Sporothrix, Stemphylium, Trichophyton, Trichosporon, and Xylohypha. Examples of protozoa include, but are not limited to, Babesia, Balantidium, Besnoitia, Cryptosporidium, Eimeria, Encephalitozoon, Entamoeba, Giardia, Hammondia, Hepatozoon, Isospora, Leishmania, Microsporidia, Neospora, Nosema, Pentatrichomonas, Plasmodium. Examples of helminth parasites include, but are not limited to, Acanthocheilonema, Aelurostrongylus, Ancylostoma, Angiostrongylus, Ascaris, Brugia, Bunostomum, Capillaria, Chabertia, Cooperia, Crenosoma, Dictyocaulus, Dioctophyme, Dipetalonema, Diphyllobothrium, Diplydium, Dirofilaria, Dracunculus, Enterobius, Filaroides, Haemonchus, Lagochilascaris, Loa, Mansonella, Muellerius, Nanophyetus, Necator, Nematodirus, Oesophagostomum, Onchocerca, Opisthorchis, Ostertagia, Parafilaria, Paragonimus, Parascaris, Physaloptera, and/or Protostrongylus.

The exosome compositions of the disclosure may be administered by any suitable means. Administration to a human or animal subject may be selected from rectal, buccal, vaginal, parenteral, intramuscular, intracerebral, intravascular (including intravenous), intracutaneous, subcutaneous, intranasal, intracardiac, intracerebroventricular, intraperitoneal intraarticular, intrasynovial, intrasternal, intrathecal, intralesional, or intracranial routes, or transdermal administration, or via an implanted reservoir.

The exosomes may be delivered as a composition. The composition may be formulated for any suitable means of administration, including rectal, buccal, vaginal, parenteral, intramuscular, intracerebral, intravascular (including intravenous), intracutaneous, subcutaneous, intranasal, intracardiac, intracerebroventricular, intraperitoneal intraarticular, intrasynovial, intrasternal, intrathecal, intralesional, or intracranial routes, or transdermal administration, or via an implanted reservoir. Compositions for parenteral administration may include sterile aqueous solutions which may also contain buffers, diluents and other suitable additives. The exosomes of the disclosure may be formulated in a pharmaceutical composition, which may include pharmaceutically acceptable carriers, thickeners, diluents, buffers, preservatives, and other pharmaceutically acceptable carriers or excipients and the like in addition to the exosomes.

A “pharmaceutically acceptable carrier” (excipient) is a pharmaceutically acceptable solvent, suspending agent or any other pharmacologically inert vehicle for delivering one or more nucleic acids to a subject. Typical pharmaceutically acceptable carriers include, but are not limited to, binding agents (e.g. pregelatinized maize starch, polyvinylpyrrolidone or hydroxypropyl methylcellulose, etc.); fillers (e.g. lactose and other sugars, microcrystalline cellulose, pectin, gelatin, calcium sulfate, ethyl cellulose, polyacrylates or calcium hydrogen phosphate, etc.); lubricants (e.g. magnesium stearate, talc, silica, colloidal silicon dioxide, stearic acid, metallic stearates, hydrogenated vegetable oils, corn starch, polyethylene glycols, sodium benzoate, sodium acetate, etc.); disintegrates (e.g. starch, sodium starch glycolate, etc.); or wetting agents (e.g.

The compositions provided herein may additionally contain other adjunct components conventionally found in pharmaceutical compositions. Thus, for example, the compositions may contain additional compatible pharmaceutically active materials or may contain additional materials useful in physically formulating various dosage forms of the composition of present invention, such as dyes, flavoring agents, preservatives, antioxidants, opacifiers, thickening agents and stabilizers. However, such materials, when added, should not unduly interfere with the biological activities of the components of the compositions provided herein.

A therapeutically effective amount of composition is administered. The therapeutically effective amount of the produced exosomes is that amount that achieves a desired effect in a subject being treated. For instance, this can be the amount of exosomes necessary to inhibit advancement, or to cause regression of, cancer, or which is capable of relieving symptoms caused by cancer. This can be the amount of exosomes necessary to inhibit advancement, or to cause regression, of an autoimmune or alloimmune disease, or which is capable of relieving symptoms caused by an autoimmune disease, such as pain and inflammation. It can also be of the amount of exosomes necessary to inhibit advancement, or to cause regression, of a microbial infection, or which is capable of relieving symptoms caused by a microbial infection.

The produced exosomes can be administered in treatment regimens consistent with the disease, for example a single or a few doses over one to several days to ameliorate a disease state or periodic doses over an extended time to inhibit disease progression and prevent disease recurrence. The dose may be determined according to various parameters, especially according to the severity of the condition, age, and weight of the patient to be treated; the route of administration; and the required regimen. A physician will be able to determine the required route of administration and dosage for any particular patient. Optimum dosages may vary depending on the relative potency of individual constructs and can generally be estimated based on ECsos found to be effective in vitro and in in vivo animal models. In general, dosage is from 0.01 mg/kg to 100 mg per kg of body weight. A typical daily dose is from about 0.1 to 50 mg per kg, preferably from about 0.1 mg/kg to 10 mg/kg of body weight, according to the potency of the specific construct, the age, weight and condition of the subject to be treated, the severity of the disease and the frequency and route of administration. Different dosages of the construct may be administered depending on whether administration is by intramuscular injection or systemic (intravenous or subcutaneous) injection. In some cases, the dose of single or multiple systemic injections is in the range of 10 to 100 mg/kg of body weight.

In some cases, the individual may have to be treated repeatedly, for example once or more daily, weekly, monthly or yearly. Persons of ordinary skill in the art can easily estimate repetition rates for dosing based on measured residence times and concentrations of the construct in bodily fluids or tissues. Following successful treatment, it may be desirable to have the individual undergo maintenance therapy, wherein the construct is administered in maintenance doses, ranging from 0.01 mg/kg to 100 mg per kg of body weight, once or more daily, to once every 20 years.

### VI. Kits

Any of the compositions described herein may be comprised in a kit. In a nonlimiting example, cells, reagents to produce cells, exosomes, and reagents to produce exosomes, and/or components thereof may be comprised in a kit. In certain embodiments, exosomes may be comprised in a kit, and they may or may not yet express one or more bioactive substances. Such a kit may or may not have one or more bioactive substances to be loaded into the exosomes, including reagents to generate same and/or reagents to manipulate the exosomes for loading of the agents. Such agents include small molecules, proteins, nucleic acids, antibodies, buffers, primers, nucleotides, salts, and/or a combination thereof, for example.

In particular embodiments, the kit comprises the exosome-based therapy of the disclosure and also another therapy. In some cases, the kit, in addition to the exosome-based therapy embodiments, also includes a second therapy, such as chemotherapy, hormone therapy, immunotherapy, and/or antimicrobial therapy, for example. The kit(s) may be tailored to a particular disease for an individual and comprise respective second therapies for the individual.

The article of manufacture or kit can further comprise a package insert comprising instructions for using the exosomes to treat or delay progression of disease, for example, cancer, an infection, or an immune disorder, in an individual or to enhance treatment of an individual having cancer, an infection, or an immune disorder. Any of the exosomes described herein may be included in the article of manufacture or kits. Suitable containers include, for example, bottles, vials, bags and syringes. The container may be formed from a variety of materials such as glass, plastic (such as polyvinyl chloride or polyolefin), or metal alloy (such as stainless steel or HASTELLOY®). In some embodiments, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some embodiments, the article of manufacture further includes one or more of another agent (e.g., a chemotherapeutic substance, an anti-neoplastic agent, an anti-microbial agent). Suitable containers for the one or more agent include, for example, bottles, vials, bags and syringes.

### EXAMPLES

It should be known by those of skill in the art that the techniques disclosed in the examples which follow represent techniques discovered by the inventor to function well in the practice of the disclosure. However, those of skill in the art should, in light of the present disclosure, appreciate that many changes can be made in the specific embodiments which are disclosed and still obtain a like or similar result without departing from the spirit and scope of the disclosure.

### Example 1

### Preparation of MSC-Exos

In some embodiments, MSC-Exos were obtained from dMSCs, preferably placental tissue-derived mesenchymal stem cells (“PL-MSCs”), and amniotic fluid of healthy human donors. PL-MSC were grown in complete DMEM. Low passage (<5) PL-MSCs were grown to 60%-80% confluence in multiflasks before isolation. Fresh PL-MSC media were layered and collected after 48 to 72 h (conditioned medium). Exosomes (“Exos”) were isolated by ultracentrifugation (100,000 g at 4° C. for 70 min). The isolation of exosomes was performed by positive selection using the μMACS™ Separator (Miltenyi Biotec, Bergisch Gladbach, Germany; Cat. No. 130-042-602) and the Exosome Isolation Kit Pan, human (Miltenyi Biotec, Bergisch Gladbach, Germany; Cat. No. 130-110-912) which contained a cocktail of MicroBeads conjugated to the tetraspanin proteins CD9, CD63, and CD81.

Umbilical cord tissue can be extremely attractive and low-cost alternative source of MSCs (UC-MSCs). Generally, UC-MSCs show a higher expansion capacity and a greater exosome yield per cell than BM-MSCs. Given the advantages of UC-MSCs over BM-MSCs as a source of exosomes, UC-Exos can be used, in some aspects, as delivery vehicles in tumor therapy, while in other aspects related to selected diseases and situations, BM exosomes can be used.

### Example 2

Exosome Electroporation to load miRNAs

One strategy for producing Exo-miRNAs relies on transducing MSCs with lentivirus (LV) containing the cDNA of the mi-RNA, followed by isolation of the mi-RNA from the supernatant. In other cases, mature miRNAs are directly loaded into exosomes by electroporation. Standard operating procedures (SOPs) were utilized for the electroporation of miRNAs into UC-Exos. Specifically, UC-MSCs were cultured, supernatant collected, and UC-Exos isolated by centrifugation. Human miRNA double stranded mature miR-mimic (Sigma Aldrich) was electroporated into UC-Exos. Each electroporation reaction contained approximately 1-2 pg of total exosomal protein.

To assess the amount of miRNA loaded into the UC-Exos, the miRNA is treated with RNase (or without RNase as control) to eliminate any free miRNA, total RNA was isolated using TRIZOL™ or the like, and RT-qPCR was performed using primers specific for a particular miRNA. Samples with known quantities of the miRNA are simultaneously assayed to develop a standard curve. Based on the results, electroporation programs that consistently have the lowest Ct value across all replicates were identified. Isolation and use of miRNAs proved very successful. For example, treatment with miR-182 significantly increased tumor toxicity. Further, administration of miR-23b to a subject induced dormancy of BM2 breast cancer cells and promoted resistance to docetaxel. In addition to loading exosomes with miRs, synthetic interfering RNAs (siRNA) can also be loaded into MSC-derived exosomes.

### Example 3

### Providing Umbilical Cord Mesenchymal Stem Cell-Derived Exosomes to Treat Toxicities

In addition to the capacity of UC-Exos to deliver bioactive substances, the potential of UC-MSCs to mitigate treatment induced CNS toxicities is also reported. Based on recent evidence indicating that exosomes are capable of reversing traumatic brain injury and inflammation, in some aspects, treatment with MSC-derived exosomes may be as effective as MSCs at reversing chemoradiation-induced brain injury. These data suggested that, in some embodiments, bioactive substances delivered by exosomes such as UC-Exos may be effective in the treatment of neurocognitive toxicities secondary to radiation and chemotherapy.

What is claimed is:

1. A method of producing exosomes, the method comprising:
modifying one or more types of mesenchymal stem cells (MSCs) to introduce one or more bioactive substances into the one or more types of MSCs, to produce modified MSCs;
culturing the modified MSCs, to produce a culture; and
collecting exosomes from the culture,
wherein the exosomes are generated from the modified MSCs.

2. The method of claim 1, further comprising:
enriching or concentrating the exosomes from the culture by differential ultracentrifugation and filtration through a sucrose gradient.

3. The method of claim 1, wherein the modifying is performed by a process selected from the group consisting of: transfection, transduction, electroporation, and combinations thereof.

4. The method of claim 1, wherein the one or more bioactive substances are exogenous with respect to the one or more types of MSCs.

5. The method of claim 1, further comprising:
testing a sample of the exosomes to determine one or more characteristics thereof.

6. The method of claim 1, wherein the one or more bioactive substances comprise a substance selected from the group consisting of: one or more chemotherapeutic agents, one or more antimicrobial agents, one or more antibiotics, one or more anti-fungal agents, one or more agents for treating an auto-immune or alloimmune disease, one or more gene-modifying components, one or more enzymes, one or more proteins, and combinations thereof.

7. A method of producing exosomes, the method comprising:
culturing one or more types of mesenchymal stem cells (MSCs), to produce a culture;
collecting exosomes from the culture;
modifying the collected exosomes to load one or more bioactive substances into the collected exosomes.

8. The method of claim 7, wherein the culturing occurs under conditions balanced with nitrogen, and wherein the method occurs in a cell culture reactor comprising a plurality of porous microchannels.

9. The method of claim 7, wherein the one or more types of MSCs are derived from a tissue selected from the group consisting of: umbilical cord tissue, umbilical cord blood, bone marrow, adipose tissue, dental tissue, placental tissue, peripheral blood, Wharton's Jelly, skin tissue, and combinations thereof.

10. The method of claim 7, wherein the one or more bioactive substances is selected from the group consisting of: deoxyribonucleic acid (DNA), ribonucleic acid (RNA), microRNA (miRNA), short hairpin RNA (shRNA), small interfering RNA (siRNA), proteins, peptides, lipids, antibodies, antibody fragments, drugs, and combinations thereof.

11. The method of claim 10, wherein the miRNA is selected from the group consisting of: miR-182, miR-23b, miR-15a, miR-21-5p, miR-124, miR-148a, miR-let-7a, miR-let-7b, miR-let-7c, miR-let-7d, miR-let-7e, miR-let-7f, miR-let-7g, miR-let-7h, miR-let-7i, miR-135a-2, miR-668, miR-942, miR-657, and combinations thereof.

12. The method of claim 7, wherein loading efficiency of the one or more bioactive substances into the collected exosomes is at least 30%.

13. The method of claim 7, further comprising:
administering to a subject an effective amount of the modified exosomes.

14. The method of claim 13, wherein, after the administering, the modified exosomes result in at least one of:
protection against one or more nervous system toxicities caused by one or more additional therapies delivered to the subject,
improve and/or reverse cognitive impairment and/or neurodegeneration caused by the one or more additional therapies delivered to the subject,
reduction of inflammation in the subject, and
regenerating and/or repairing one or more tissues in the subject.

15. The method of claim 14, wherein the one or more additional therapies are selected from the group consisting of: chemotherapy, radiation therapy, and combinations thereof.

16. A composition for delivering exosomes into one or more tumor cells, comprising:
one or more exosomes derived from one or more types of mesenchymal stem cells (MSCs); and
one or more bioactive substances located within the one or more exosomes.

17. The composition of claim 16, wherein the one or more exosomes are isolated from autologous cells of a subject.

18. The composition of claim 16, wherein the one or more bioactive substances comprises one or more plasmids.

19. The composition of claim 18, wherein the one or more plasmids are selected from the group consisting of: a deoxynucleic acid (DNA) plasmid, a ribonucleic acid (RNA) plasmid, a retrovirus, an adeno-associated virus (AAV), and combinations thereof.

20. The composition of claim 18, wherein the one or more plasmids promote expression of one or more vascular endothelial growth factor (VEGF) genes.
